(19) United States
(12) Patent Application Publication
(10) Pub. No.: US 2023/0136203 A1
(43) Pub. Date: May 4, 2023

(54) METHODS OF TREATING HER2 MUTANT CANCERS WITH TUCATINIB

(71) Applicant: Seagen Inc., Bothell, WA (US)

(72) Inventor: Scott PETERSON, Bothell, WA (US)

(21) Appl. No.: 17/910,265

(22) PCT Filed: Mar. 9, 2021
(86) PCT No.: PCT/US2021/021527

§ 371 Date: Sep. 8, 2022

Related U.S. Application Data
(60) Provisional application No. 62/988,312, filed on Mar. 11, 2020.

(51) Int. Cl. A61K 31/517; A61P 35/00; A61K 31/7068; C07K 16/32

(52) U.S. Cl. CPC A61K 31/517; A61P 35/00; A61K 31/7068; C07K 16/32; A61K 2039/505

(57) ABSTRACT

The invention provides methods of treating cancer, such as cancers with a HER2 mutation, with tucatinib, or salt or solvate thereof. The invention also provides compositions and kits comprising tucatinib for use in treating cancer, such as cancers with a HER2 mutation.

### CROSS-REFERENCE TO RELATED APPLICATIONS

This application claims priority to U.S. Provisional Application No. 62/988,312 filed on Mar. 11, 2020, the content of which is incorporated herein by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 761682003540SEQLIST.TXT, date recorded: Mar. 1, 2021, size: 11 KB).

### TECHNICAL FIELD

The present invention relates to methods of treating cancer, such as cancers with a HER2 mutation, with tucatinib, or salt or solvate thereof.

### BACKGROUND

HER2 (human epidermal growth factor receptor 2)/ErbB2/Neu is a member of the epidermal growth factor receptor (EGFR) family of homologous transmembrane receptor tyrosine kinases (EGFR and HER2-4/ErbB1-4). Ligand binding to EGFR or HER3/4 induces a conformational change in these proteins that facilitates receptor dimerization. Receptor dimerization brings the two intracellular tyrosine kinase domains (TKDs) together in an asymmetrical manner and the carboxy lobe of one allosterically activates the amino lobe of the other. Subsequent transphosphorylation of tyrosines in the carboxy tail provides docking sites for the recruitment of downstream signaling proteins. These signaling proteins affect multiple cellular processes, including proliferation, survival and differentiation, depending on receptor subtype and cellular context.

Amplification or over-expression of this oncogene has been shown to play an important role in the development and progression of certain aggressive types of breast cancer. In recent years, the protein has become an important biomarker and target of therapy for approximately 30% of breast cancer patients.

Tucatinib is an orally bioavailable, small molecule tyrosine kinase inhibitor (TKI) that is highly selective for HER2, a growth factor receptor that is over-expressed in multiple cancers, including breast, colorectal, and gastric cancers. Between 15% and 20% of breast cancers cases worldwide are HER2-positive.

In typical HER2+ cancers, including breast cancer, gastric cancer, and colorectal cancer, the amplification of HER2 leads to strong signal transduction through either homodimerization or heterodimerization with another ErbB-family member. This results in downstream activation of both the MAP kinase and phosphatidyl-inositol-3 (PI3) kinase pathways, which in turn enhances mitogenicity and survival.

In some cancers, however, HER2 expression is not amplified, but rather HER2 may contain an activating mutation in the kinase domain that also leads to increased signaling and mitogenicity. See WO 2018/200505. HER2 activating mutations may act as oncogenic drivers in various cancer types. See WO 2018/200505. In the clinic, they can be identified by next generation sequencing (NGS) in either tumor biopsies or circulating cell-free DNA (cfDNA). Annals of Oncol 28:136-141 (2017). Preclinical data indicate that HER2 “hot spot” mutations may be constitutively active, have transforming capacity in vitro and in vivo and may show variable sensitivity to anti-HER2 based therapies. J Mol Diagn, 17(5):487-495 (2015), Nat Gen 51, 207-216 (2019). Recent clinical trials also revealed potential activity of HER2-targeted drugs against a variety of tumors harboring HER2 mutations. HER2-targeted agents could potentially be useful for the treatment of cancers harboring these activating mutations. ESMO Open 2017; 2: e000279. However, efforts to target cancers with HER2 mutations have met with limited clinical success, possibly because of their low frequency, inadequate understanding of the biological activity of these mutations, and difficulty in separating the drivers from the passenger mutations. The Oncologist 24(12):e1303-e1314 (2019).

All references cited herein, including patent applications, patent publications, and scientific literature, are herein incorporated by reference in their entirety, as if each individual reference were specifically and individually indicated to be incorporated by reference.

### SUMMARY

Provided herein is a method for treating cancer in a subject comprising administering a therapeutically effective amount of tucatinib, or salt or solvate thereof, to the subject, wherein the cancer has been determined to express a mutant form of HER2. Also provided herein is a method for treating cancer in a subject comprising administering a therapeutically effective amount of tucatinib, or salt or solvate thereof, to the subject, wherein the cancer expresses a mutant form of HER2. In some embodiments, the mutant form of HER2 is determined by DNA sequencing. In some embodiments, the mutant form of HER2 is determined by determining RNA sequencing. In some embodiments, the mutant form of HER2 is determined by nucleic acid sequencing. In some embodiments, the nucleic acid sequencing is next-generation sequencing (NGS). In some embodiments, the mutant form of HER2 is determined by polymerase chain reaction (PCR). In some embodiments, the mutant form of HER2 is determined by analyzing a sample obtained from the subject. In some embodiments, the sample obtained from the subject is a cell-free plasma sample. In some embodiments, the sample obtained from the subject is a tumor biopsy. In some embodiments, the cancer does not have HER2 amplification and the absence of HER2 amplification is determined by immunohistochemistry (IHC). In some embodiments, the cancer has a HER2 amplification score of 0 or 1+ and the HER2 amplification score is determined by immunohistochemistry (IHC). In some embodiments, the cancer has less than a 2 fold increase in HER2 protein levels. In some embodiments, the mutant form of HER2 comprises at least one amino acid substitution, insertion, or deletion compared to the amino acid sequence of SEQ ID NO:1. In some embodiments, the mutation in HER2 is an activating mutation. In some embodiments, the mutant form of HER2 comprises the amino acid substitution L755S. In some embodiments, the mutant form of HER2 comprises the amino acid substitution V777L. In some embodiments, the mutant form of HER2 comprises the amino acid substitution S310Y. In some embodiments, the mutant form of HER2 comprises a G776 YVMA insertion (G776 ins YVMA). In some embodiments, the cancer is selected from the group consisting of gastric cancer, colorectal cancer, lung cancer, gall bladder cancer, and breast cancer. In some embodiments, the lung cancer is non-small cell lung cancer. In some embodiments, the breast cancer is a HER2 positive breast cancer. In some embodiments, the tucatinib, or salt or solvate thereof, is administered to the subject at a dose of about 150 mg to about 650 mg. In some embodiments, the tucatinib, or salt or solvate thereof, is administered to the subject at a dose of about 300 mg. In some embodiments, the tucatinib, or salt or solvate thereof, is administered once or twice per day. In some embodiments, the tucatinib, or salt or solvate thereof, is administered to the subject at a dose of about 300 mg twice per day. In some embodiments, the tucatinib is administered to the subject orally. In some embodiments, the method further comprises administering one or more additional therapeutic agents to the subject to treat the cancer. In some embodiments, the one or more additional therapeutic agents is selected from the group consisting of capecitabine and an anti-HER2 antibody. In some embodiments, the one or more additional therapeutic agents is capecitabine. In some embodiments, the one or more additional therapeutic agents is trastuzumab. In some embodiments, the one or more additional therapeutic agents are capecitabine and trastuzumab. In some embodiments, the capecitabine is administered to the subject at a dose of about 500 mg/m² to about 1500 mg/m². In some embodiments, the capecitabine is administered to the subject at a dose of about 1000 mg/m². In some embodiments, the capecitabine is administered to the subject orally. In some embodiments, the capecitabine is administered to the subject twice per day. In some embodiments, the trastuzumab is administered to the subject at a dose of about 400 mg to about 800 mg. In some embodiments, the trastuzumab is administered to the subject at a dose of about 600 mg. In some embodiments, the trastuzumab is administered to the subject subcutaneously. In some embodiments, the trastuzumab is administered to the subject intraperitoneally. In some embodiments, the trastuzumab is administered to the subject at a dose of about 4 mg/kg to about 10 mg/kg. In some embodiments, the trastuzumab is administered to the subject at a dose of about 6 mg/kg. In some embodiments, the trastuzumab is administered to the subject at a dose of about 8 mg/kg. In some embodiments, the trastuzumab is administered to the subject at an initial dose of about 8 mg/kg followed by subsequent doses of about 6 mg/kg. In some embodiments, the trastuzumab is administered intravenously. In some embodiments, the trastuzumab is administered once about every 1 week, once about every 2 weeks, once about every 3 weeks, or once about every 4 weeks. In some embodiments, the trastuzumab is administered once about every 3 weeks. In some embodiments, the tucatinib, capecitabine and trastuzumab are administered to the subject on a 21 day treatment cycle. In some embodiments, the tucatinib is administered to the subject twice per day on each day of the 21 day treatment cycle. In some embodiments, the capecitabine is administered to the subject twice per day on each of days 1-14 of the 21 day treatment cycle. In some embodiments, the trastuzumab is administered to the subject once per 21 day treatment cycle. In some embodiments, the dose of trastuzumab during the first 21 day treatment cycle is 8 mg/kg and the dose of trastuzumab during the subsequent 21 day treatment cycles is 6 mg/kg. In some embodiments, treating the subject results in a tumor growth inhibition (TGI) index of at least about 85%. In some embodiments, treating the subject results in a TGI index of about 100%. In some embodiments, one or more therapeutic effects in the subject is improved after administration of tucatinib to the subject relative to a baseline. In some embodiments, the one or more therapeutic effects is selected from the group consisting of: size of a tumor derived from the cancer, objective response rate, duration of response, time to response, progression free survival and overall survival. In some embodiments, the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of tucatinib to the subject. In some embodiments, the objective response rate is at least about 20%, at least about 25%, at least about 30%, at least about 35%, at least about 40%, at least about 45%, at least about 50%, at least about 60%, at least about 70%, or at least about 80%. In some embodiments, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of tucatinib to the subject. In some embodiments, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of tucatinib to the subject. In some embodiments, the duration of response to tucatinib is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of tucatinib to the subject. In some embodiments, the subject is a human.

Also provided herein is the use of a therapeutically effective amount of tucatinib, or salt or solvate thereof, for the manufacture of a medicament for use according to any of the embodiments herein.

Also provided herein is tucatinib, or a salt or solvate thereof, for use according to any of the embodiments herein.

Also provided herein is a kit comprising tucatinib, or salt or solvate thereof, and instructions for using the kit according to any of the embodiments herein.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a graph showing mean (±SEM) tumor volume over time of mice treated with vehicle, tucatinib, trastuzumab, or a combination of tucatinib and trastuzumab in the GL1208 gallbladder cancer xenograft model.

FIG. 2A is a graph showing mean (±SEM) tumor volume over time of mice treated with vehicle, tucatinib, trastuzumab, or a combination of tucatinib and trastuzumab in the CR3056 colorectal cancer xenograft model. FIG. 2B is a graph showing tucatinib mediated inhibition of HER2 V777L kinase activity.

FIG. 3 is a graph showing mean (±SEM) tumor volume over time of mice treated with vehicle, tucatinib, trastuzumab, or a combination of tucatinib and trastuzumab in the GA2140 gastric cancer xenograft model.

FIG. 4 is a graph showing mean (±SEM) tumor volume over time of mice treated with vehicle, tucatinib, trastuzumab, or a combination of tucatinib and trastuzumab in the GA6210 gastric cancer xenograft model.

FIG. 5A is a graph showing mean (±SEM) tumor volume over time of mice treated with vehicle, tucatinib, trastuzumab, or a combination of tucatinib and trastuzumab in the LU-5239 non-small cell lung cancer xenograft model. FIG. 5B is a graph showing tucatinib mediated inhibition of HER2 L755S kinase activity.

FIG. 6 is a graph showing mean (±SEM) tumor volume over time of mice treated with vehicle, tucatinib, trastuzumab, or a combination of tucatinib and trastuzumab in the CR-5085 colorectal cancer xenograft model.

FIG. 7A is a graph showing mean (±SEM) tumor volume over time of mice treated with vehicle, tucatinib, trastuzumab, or a combination of tucatinib and trastuzumab in a G776insYVMA non-small cell lung cancer xenograft model. FIG. 7B is a graph showing tucatinib mediated inhibition of HER2 G776insYVMA kinase activity.

### DETAILED DESCRIPTION

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “or” as used herein should in general be construed non-exclusively. For example, a claim to “a composition comprising A or B” would typically present an aspect with a composition comprising both A and B. “Or” should, however, be construed to exclude those aspects presented that cannot be combined without contradiction (e.g., a composition pH that is between 9 and 10 or between 7 and 8).

The group “A or B” is typically equivalent to the group “selected from the group consisting of A and B.”

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that aspects and embodiments of the invention described herein include “comprising,” “consisting,” and “consisting essentially of” aspects and embodiments.

The terms “about” and “approximately” as used herein shall generally mean an acceptable degree of error for the quantity measured given the nature or precision of the measurements. Typical, exemplary degrees of error are within 20 percent (%), preferably within 10%, and more preferably within 5% of a given value or range of values. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and provide written description support for a claim limitation of, e.g., “0.98X.” The terms “about” and “approximately,” particularly in reference to a given quantity, encompass and describe the given quantity itself.

Alternatively, in biological systems, the terms “about” and “approximately” may mean values that are within an order of magnitude, preferably within 5-fold, and more preferably within 2-fold of a given value. Numerical quantities given herein are approximate unless stated otherwise, meaning that the term “about” or “approximately” can be inferred when not expressly stated.

When “about” is applied to the beginning of a numerical range, it applies to both ends of the range. Thus, “from about 5 to 20%” is equivalent to “from about 5% to about 20%.” When “about” is applied to the first value of a set of values, it applies to all values in that set. Thus, “about 7, 9, or 11 mg/kg” is equivalent to “about 7, about 9, or about 11 mg/kg.”

The term “comprising” as used herein should in general be construed as not excluding additional ingredients. For example, a claim to “a composition comprising A” would cover compositions that include A and B; A, B, and C; A, B, C, and D; A, B, C, D, and E; and the like.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

As used herein, the term “co-administering” includes sequential or simultaneous administration of two or more structurally different compounds. For example, two or more structurally different pharmaceutically active compounds can be co-administered by administering a pharmaceutical composition adapted for oral administration that contains two or more structurally different active pharmaceutically active compounds. As another example, two or more structurally different compounds can be co-administered by administering one compound and then administering the other compound. The two or more structurally different compounds can be comprised of an anti-HER2 antibody and tucatinib. In some instances, the co-administered compounds are administered by the same route. In other instances, the co-administered compounds are administered via different routes. For example, one compound can be administered orally, and the other compound can be administered, e.g., sequentially or simultaneously, via intravenous, intramuscular, subcutaneous, or intraperitoneal injection. The simultaneously or sequentially administered compounds or compositions can be administered such that an anti-HER2 antibody and tucatinib are simultaneously present in a subject or in a cell at an effective concentration.

A “cancer” refers to a broad group of various diseases characterized by the uncontrolled growth of abnormal cells in the body. A “cancer” or “cancer tissue” can include a tumor. Unregulated cell division and growth results in the formation of malignant tumors that invade neighboring tissues and can also metastasize to distant parts of the body through the lymphatic system or bloodstream. Following metastasis, the distal tumors can be said to be “derived from” the pre-metastasis tumor. For example, a “tumor derived from” a breast cancer refers to a tumor that is the result of a metastasized breast cancer.

In the context of cancer, the term “stage” refers to a classification of the extent of cancer. Factors that are considered when staging a cancer include but are not limited to tumor size, tumor invasion of nearby tissues, and whether the tumor has metastasized to other sites. The specific criteria and parameters for differentiating one stage from another can vary depending on the type of cancer. Cancer staging is used, for example, to assist in determining a prognosis or identifying the most appropriate treatment option(s).

One non-limiting example of a cancer staging system is referred to as the “TNM” system. In the TNM system, “T” refers to the size and extent of the main tumor, “N” refers to the number of nearby lymph nodes to which the cancer has spread, and “M” refers to whether the cancer has metastasized. “TX” denotes that the main tumor cannot be measured, “TO” denotes that the main tumor cannot be found, and “T1,” “T2,” “T3,” and “T4” denote the size or extent of the main tumor, wherein a larger number corresponds to a larger tumor or a tumor that has grown into nearby tissues. “NX” denotes that cancer in nearby lymph nodes cannot be measured, “NO” denotes that there is no cancer in nearby lymph nodes, and “N1,” “N2,” “N3,” and “N4” denote the number and location of lymph nodes to which the cancer has spread, wherein a larger number corresponds to a greater number of lymph nodes containing the cancer. “MX” denotes that metastasis cannot be measured, “M0” denotes that no metastasis has occurred, and “M1” denotes that the cancer has metastasized to other parts of the body.

As another non-limiting example of a cancer staging system, cancers are classified or graded as having one of five stages: “Stage 0,” “Stage I,” “Stage II,” “Stage III,” or “Stage IV.” Stage 0 denotes that abnormal cells are present, but have not spread to nearby tissue. This is also commonly called carcinoma in situ (CIS). CIS is not cancer, but may subsequently develop into cancer. Stages I, II, and III denote that cancer is present. Higher numbers correspond to larger tumor sizes or tumors that have spread to nearby tissues. Stage IV denotes that the cancer has metastasized. One of skill in the art will be familiar with the different cancer staging systems and readily be able to apply or interpret them.

The term “HER2” (also known as also known as HER2/neu, ERBB2, CD340, receptor tyrosine-protein kinase erbB-2, proto-oncogene Neu, and human epidermal growth factor receptor 2) refers to a member of the human epidermal growth factor receptor (HER/EGFR/ERBB) family of receptor tyrosine kinases. Amplification or overexpression of HER2 plays a significant role in the development and progression of certain aggressive types of cancer, including colorectal cancer, gastric cancer, lung cancer (e.g., non-small cell lung cancer (NSCLC)), biliary cancers (e.g., cholangiocarcinoma, gallbladder cancer), bladder cancer, esophageal cancer, melanoma, ovarian cancer, liver cancer, prostate cancer, pancreatic cancer, small intestine cancer, head and neck cancer, uterine cancer, cervical cancer, and breast cancer. Non-limiting examples of HER2 nucleotide sequences are set forth in GenBank reference numbers NP_001005862, NP_001289936, NP_001289937, NP_001289938, and NP_004448. Non-limiting examples of HER2 peptide sequences are set forth in GenBank reference numbers NP_001005862, NP_001276865, NP_001276866, NP_001276867, and NP_004439.

When HER2 is amplified or overexpressed in or on a cell, the cell is referred to as being “HER2 positive.” The level of HER2 amplification or overexpression in HER2 positive cells is commonly expressed as a score ranging from 0 to 3 (i.e., HER2 0, HER2 1+, HER2 2+, or HER2 3+), with higher scores corresponding to greater degrees of expression. Mol Biol Int. 2014:852748 (2014). The scoring method may be based on the cell membrane staining pattern as determined by immunohistochemistry and is as follows:

i. 3+: positive HER2 expression, uniform intense membrane staining of more than 30% of invasive tumor cells;

ii. 2+: equivocal for HER2 protein expression, complete membrane staining that is either nonuniform or weak in intensity but has circumferential distribution in at least 10% of cells;

iii. 0 or 1+: negative for HER2 protein expression.

The term “tucatinib,” also known as ONT-380 and ARRY-380, refers to the small molecule tyrosine kinase inhibitor that suppresses or blocks HER2 activation. Tucatinib has the following structure:

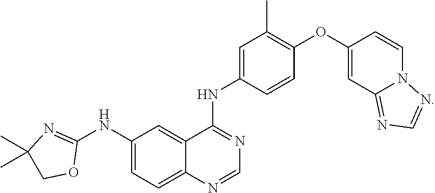 [SMILES: Cc1cc(Nc2ncnc3ccc(NC4=NC(C)(C)CO4)cc23)ccc1Oc1ccn2ncnc2c1]

The term “anti-HER2 antibody” refers to an antibody that binds to the HER2 protein. Anti-HER2 antibodies used for the treatment of cancer are typically monoclonal, although polyclonal antibodies are not excluded by the term. Anti-HER2 antibodies inhibit HER2 activation or downstream signaling by various mechanisms. As non-limiting examples, anti-HER2 antibodies can prevent ligand binding, receptor activation or receptor signal propagation, result in reduced HER2 expression or localization to the cell surface, inhibit HER2 cleavage, or induce antibody-mediated cytotoxicity. Non-limiting examples of anti-HER2 antibodies that are suitable for use in the methods and compositions of the present invention include trastuzumab, pertuzumab, ado-trastuzumab emtansine (also known as T-DM1), margetuximab, and combinations thereof.

The term “tumor growth inhibition (TGI) index” refers to a value used to represent the degree to which an agent (e.g., tucatinib, capecitabine, an anti-HER2 antibody, or a combination thereof) inhibits the growth of a tumor when compared to an untreated control. The TGI index is calculated for a particular time point (e.g., a specific number of days into an experiment or clinical trial) according to the following formula:

$$T G I = 1 - ( \frac{{Volume}_{treated ( Tx Day X )} - {Volume}_{treated ( Tx Day 0 )}}{{Volume}_{control ( Tx Day X )} - {Volume}_{ control ( Tx Day 0 )}} ) \times 100 \% ,$$

where “Tx Day 0” denotes the first day that treatment is administered (i.e., the first day that an experimental therapy or a control therapy (e.g., vehicle only) is administered) and “Tx Day X” denotes X number of days after Day 0. Typically, mean volumes for treated and control groups are used. As a non-limiting example, in an experiment where study day 0 corresponds to “Tx Day 0” and the TGI index is calculated on study day 28 (i.e., “Tx Day 28”), if the mean tumor volume in both groups on study day 0 is 250 mm³ and the mean tumor volumes in the experimental and control groups are 125 mm³ and 750 mm³, respectively, then the TGI index on day 28 is 125%.

As used herein, the term “synergistic” or “synergy” refers to a result that is observed when administering a combination of components or agents (e.g., a combination of tucatinib and an anti-HER2 antibody) produces an effect (e.g., inhibition of tumor growth, prolongation of survival time) that is greater than the effect that would be expected based on the additive properties or effects of the individual components. In some embodiments, synergism is determined by performing a Bliss analysis (see, e.g., Foucquier et al. Pharmacol. Res. Perspect. (2015) 3(3):e00149; hereby incorporated by reference in its entirety for all purposes). The Bliss Independence model assumes that drug effects are outcomes of probabilistic processes, and assumes that the drugs act completely independently (i.e., the drugs do not interfere with one another (e.g., the drugs have different sites of action) but each contributes to a common result). According to the Bliss Independence model, the predicted effect of a combination of two drugs is calculated using the formula:

E_{AB}E_{A}+E_{g}E_{A}×E_{B},

where E_{A} and E_{B} represent the effects of drugs A and B, respectively, and E_{AB} represents the effect of a combination of drugs A and B. When the observed effect of the combination is greater than the predicted effect E_{AB}, then the combination of the two drugs is considered to be synergistic. When the observed effect of the combination is equal to E_{AB}, then the effect of the combination of the two drugs is considered to be additive. Alternatively, when the observed effect of the combination is less than E_{AB}, then the combination of the two drugs is considered to be antagonistic.

The observed effect of a combination of drugs can be based on, for example, the TGI index, tumor size (e.g., volume, mass), an absolute change in tumor size (e.g., volume, mass) between two or more time points (e.g., between the first day a treatment is adminstered and a particular number of days after treatment is first administered), the rate of change of tumor size (e.g., volume, mass) between two or more time points (e.g., between the first day a treatment is adminstered and a particular number of days after treatment is first administered), or the survival time of a subject or a population of subjects. When the TGI index is taken as a measure of the observed effect of a combination of drugs, the TGI index can be determined at one or more time points. When the TGI index is determined at two or more time points, in some instances the mean or median value of the multiple TGI indices can be used as a measure of the observed effect. Furthermore, the TGI index can be determined in a single subject or a population of subjects. When the TGI index is determined in a population, the mean or median TGI index in the population (e.g., at one or more time points) can be used as a measure of the observed effect. When tumor size or the rate of tumor growth is used as a measure of the observed effect, the tumor size or rate of tumor growth can be measured in a subject or a population of subjects. In some instances, the mean or median tumor size or rate of tumor growth is determined for a subject at two or more time points, or among a population of subjects at one or more time points. When survival time is measured in a population, the mean or median survival time can be used as a measure of the observed effect.

The predicted combination effect E_{AB} can be calculated using either a single dose or multiple doses of the drugs that make up the combination (e.g., tucatinib and an anti-HER2 antibody). In some embodiments, the predicted combination effect E_{AB} is calculated using only a single dose of each drug A and B (e.g., tucatinib and an anti-HER2 antibody), and the values E_{A} and E_{B} are based on the observed effect of each drug when administered as a single agent. When the values for E_{A} and E_{B} are based on the observed effects of administering drugs A and B as single agents, E_{A} and E_{B} can be based on, for example, TGI indices, tumor sizes (e.g., volume, mass) measured at one or more time points, absolute changes in tumor size (e.g., volume, mass) between two or more time points (e.g., between the first day a treatment is adminstered and a particular number of days after treatment is first administered), the rates of change of tumor sizes (e.g., volume, mass) between two or more time points (e.g., between the first day a treatment is adminstered and a particular number of days after treatment is first administered), or the survival time of a subject or a population of subjects in each treatment group.

When TGI indices are taken as a measure of the observed effects, the TGI indices can be determined at one or more time points. When TGI indices are determined at two or more time points, in some instances the mean or median values can be used as measures of the observed effects. Furthermore, the TGI indices can be determined in a single subject or a population of subjects in each treatment group. When the TGI indices are determined in populations of subjects, the mean or median TGI indices in each population (e.g., at one or more time points) can be used as measures of the observed effects. When tumor sizes or the rates of tumor growth are used as measures of the observed effects, the tumor sizes or rates of tumor growth can be measured in a subject or a population of subjects in each treatment group. In some instances, the mean or median tumor sizes or rates of tumor growth are determined for subjects at two or more time points, or among populations of subjects at one or more time points. When survival time is measured in a population, mean or median survival times can be used as measures of the observed effects.

In some embodiments, the predicted combination effect E_{AB} is calculated using a range of doses (i.e., the effects of each drug, when administered as a single agent, are observed at multiple doses and the observed effects at the multiple doses are used to determine the predicted combination effect at a specific dose). As a non-limiting example, E_{AB} can be calculated using values for E_{A} and E_{B} that are calculated according to the following formulae:

$${E}_{A} = {E}_{A max} \times \frac{{a}^{p}}{{A}_{50}^{p} + {a}^{p}}$$

$${E}_{B} = {E}_{B max} \times \frac{{b}^{q}}{{B}_{50}^{q} + {b}^{q}} ,$$

where E_{Amax} and E_{Bmax} are the maximum effects of drugs A and B, respectively, A₅₀ and B₅₀ are the half maximum effective doses of drugs A and B, respectively, a and b are administered doses of drugs A and B, respectively, and p and q are coefficients that are derived from the shapes of the dose-response curves for drugs A and B, respectively (see, e.g., Foucquier et al. Pharmacol. Res. Perspect. (2015) 3(3):e00149).

In some embodiments, a combination of two or more drugs is considered to be synergistic when the combination produces an observed TGI index that is greater than the predicted TGI index for the combination of drugs (e.g., when the predicted TGI index is based upon the assumption that the drugs produced a combined effect that is additive). In some instances, the combination is considered to be synergistic when the observed TGI index is at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, or 80% greater than the predicted TGI index for the combination of drugs.

In some embodiments, the rate of tumor growth (e.g., the rate of change of the size (e.g., volume, mass) of the tumor) is used to determine whether a combination of drugs is synergistic (e.g., the combination of drugs is synergistic when the rate of tumor growth is slower than would be expected if the combination of drugs produced an additive effect). In other embodiments, survival time is used to determine whether a combination of drugs is synergistic (e.g., a combination of drugs is synergistic when the survival time of a subject or population of subjects is longer than would be expected if the combination of drugs produced an additive effect).

“Treatment” or “therapy” of a subject refers to any type of intervention or process performed on, or the administration of an active agent to, the subject with the objective of reversing, alleviating, ameliorating, inhibiting, slowing down, or preventing the onset, progression, development, severity, or recurrence of a symptom, complication, condition, or biochemical indicia associated with a disease. In some embodiments, the disease is cancer.

A “subject” includes any human or non-human animal. The term “non-human animal” includes, but is not limited to, vertebrates such as non-human primates, sheep, dogs, and rodents such as mice, rats, and guinea pigs. In some embodiments, the subject is a human. The terms “subject” and “patient” and “individual” are used interchangeably herein.

An “effective amount” or “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

By way of example for the treatment of tumors, a therapeutically effective amount of an anti-cancer agent inhibits cell growth or tumor growth by at least about 10%, by at least about 20%, by at least about 30%, by at least about 40%, by at least about 50%, by at least about 60%, by at least about 70%, or by at least about 80%, by at least about 90%, by at least about 95%, by at least about 96%, by at least about 97%, by at least about 98%, or by at least about 99% in a treated subject(s) (e.g., one or more treated subjects) relative to an untreated subject(s) (e.g., one or more untreated subjects). In some embodiments, a therapeutically effective amount of an anti-cancer agent inhibits cell growth or tumor growth by 100% in a treated subject(s) (e.g., one or more treated subjects) relative to an untreated subject(s) (e.g., one or more untreated subjects).

In other embodiments of the disclosure, tumor regression can be observed and continue for a period of at least about 20 days, at least about 30 days, at least about 40 days, at least about 50 days, or at least about 60 days.

A therapeutically effective amount of a drug (e.g., tucatinib) includes a “prophylactically effective amount,” which is any amount of the drug that, when administered alone or in combination with an anti-cancer agent to a subject at risk of developing a cancer (e.g., a subject having a pre-malignant condition) or of suffering a recurrence of cancer, inhibits the development or recurrence of the cancer. In some embodiments, the prophylactically effective amount prevents the development or recurrence of the cancer entirely. “Inhibiting” the development or recurrence of a cancer means either lessening the likelihood of the cancer's development or recurrence, or preventing the development or recurrence of the cancer entirely.

As used herein, “subtherapeutic dose” means a dose of a therapeutic compound (e.g., tucatinib) that is lower than the usual or typical dose of the therapeutic compound when administered alone for the treatment of a hyperproliferative disease (e.g., cancer).

By way of example, an “anti-cancer agent” promotes cancer regression in a subject. In some embodiments, a therapeutically effective amount of the drug promotes cancer regression to the point of eliminating the cancer. “Promoting cancer regression” means that administering an effective amount of the drug, alone or in combination with an anti-cancer agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity or other adverse physiological effects at the cellular, organ and/or organism level (adverse effects) resulting from administration of the drug.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration that is at least the same as the treatment duration, or at least 1.5, 2.0, 2.5, or 3 times longer than the treatment duration.

As used herein, “complete response” or “CR” refers to disappearance of all target lesions; “partial response” or “PR” refers to at least a 30% decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for PD, taking as reference the smallest SLD since the treatment started.

As used herein, “progression free survival” or “PFS” refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall response rate” or “ORR” refers to the sum of complete response (CR) rate and partial response (PR) rate.

As used herein, “overall survival” or “OS” refers to the percentage of individuals in a group who are likely to be alive after a particular duration of time.

The term “weight-based dose”, as referred to herein, means that a dose administered to a subject is calculated based on the weight of the subject. For example, when a subject with 60 kg body weight requires 6.0 mg/kg of an agent, such as trastuzumab, one can calculate and use the appropriate amount of the agent (i.e., 360 mg) for administration to said subject.

The use of the term “fixed dose” with regard to a method of the disclosure means that two or more different agents (e.g., tucatinb and anti-HER2 antibody) are administered to a subject in particular (fixed) ratios with each other. In some embodiments, the fixed dose is based on the amount (e.g., mg) of the agents. In certain embodiments, the fixed dose is based on the concentration (e.g., mg/ml) of the agents. For example, a 1:2 ratio of tucatinib to an anti-HER2 antibody administered to a subject can mean about 300 mg of tucatinib and about 600 mg of the anti-HER2 antibody or about 3 mg/ml of tucatinib and about 6 mg/ml of the anti-HER2 antibody are administered to the subject.

The use of the term “flat dose” with regard to the methods and dosages of the disclosure means a dose that is administered to a subject without regard for the weight or body surface area (BSA) of the subject. The flat dose is therefore not provided as a mg/kg dose, but rather as an absolute amount of the agent (e.g., tucatinib or anti-HER2 antibody). For example, a subject with 60 kg body weight and a subject with 100 kg body weight would receive the same dose of tucatinb (e.g., 300 mg).

The phrase “pharmaceutically acceptable” indicates that the substance or composition must be compatible chemically and/or toxicologically, with the other ingredients comprising a formulation, and/or the mammal being treated therewith.

As used herein, the term “pharmaceutically acceptable carrier” refers to a substance that aids the administration of an active agent to a cell, an organism, or a subject. “Pharmaceutically acceptable carrier” refers to a carrier or excipient that can be included in the compositions of the invention and that causes no significant adverse toxicological effect on the subject. Non-limiting examples of pharmaceutically acceptable carriers include water, NaCl, normal saline solutions, lactated Ringer's, normal sucrose, normal glucose, binders, fillers, disintegrants, lubricants, coatings, sweeteners, flavors and colors, liposomes, dispersion media, microcapsules, cationic lipid carriers, isotonic and absorption delaying agents, and the like. The carrier may also be substances for providing the formulation with stability, sterility and isotonicity (e.g., antimicrobial preservatives, antioxidants, chelating agents and buffers), for preventing the action of microorganisms (e.g. antimicrobial and antifungal agents, such as parabens, chlorobutanol, phenol, sorbic acid and the like) or for providing the formulation with an edible flavor etc. In some instances, the carrier is an agent that facilitates the delivery of a small molecule drug or antibody to a target cell or tissue. One of skill in the art will recognize that other pharmaceutical carriers are useful in the present invention.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, pamoate (i.e., 4,4′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

“Administering” or “administration” refer to the physical introduction of a therapeutic agent to a subject, using any of the various methods and delivery systems known to those skilled in the art. Exemplary routes of administration include oral, intravenous, intramuscular, subcutaneous, intraperitoneal, spinal or other parenteral routes of administration, for example by injection or infusion (e.g., intravenous infusion). The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent can be administered via a non-parenteral route, or orally. Other non-parenteral routes include a topical, epidermal or mucosal route of administration, for example, intranasally, vaginally, rectally, sublingually or topically. Administration can also be performed, for example, once, a plurality of times, and/or over one or more extended periods.

The terms “baseline” or “baseline value” used interchangeably herein can refer to a measurement or characterization of a symptom before the administration of the therapy or at the beginning of administration of the therapy. The baseline value can be compared to a reference value in order to determine the reduction or improvement of a symptom of a disease contemplated herein (e.g., cancer). The terms “reference” or “reference value” used interchangeably herein can refer to a measurement or characterization of a symptom after administration of the therapy. The reference value can be measured one or more times during a dosage regimen or treatment cycle or at the completion of the dosage regimen or treatment cycle. A “reference value” can be an absolute value; a relative value; a value that has an upper and/or lower limit; a range of values; an average value; a median value: a mean value; or a value as compared to a baseline value.

Similarly, a “baseline value” can be an absolute value; a relative value; a value that has an upper and/or lower limit; a range of values; an average value; a median value; a mean value; or a value as compared to a reference value. The reference value and/or baseline value can be obtained from one individual, from two different individuals or from a group of individuals (e.g., a group of two, three, four, five or more individuals).

The term “monotherapy” as used herein means that the tucatinib, or salt or solvate thereof, is the only anti-cancer agent administered to the subject during the treatment cycle. Other therapeutic agents, however, can be administered to the subject. For example, anti-inflammatory agents or other agents administered to a subject with cancer to treat symptoms associated with cancer, but not the underlying cancer itself, including, for example inflammation, pain, weight loss, and general malaise, can be administered during the period of monotherapy.

An “adverse event” (AE) as used herein is any unfavorable and generally unintended or undesirable sign (including an abnormal laboratory finding), symptom, or disease associated with the use of a medical treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

A “serious adverse event” or “SAE” as used herein is an adverse event that meets one of the following criteria:
Is fatal or life-threatening (as used in the definition of a serious adverse event, “life-threatening” refers to an event in which the patient was at risk of death at the time of the event; it does not refer to an event which hypothetically might have caused death if it was more severe.
Results in persistent or significant disability/incapacity
Constitutes a congenital anomaly/birth defect
Is medically significant, i.e., defined as an event that jeopardizes the patient or may require medical or surgical intervention to prevent one of the outcomes listed above. Medical and scientific judgment must be exercised in deciding whether an AE is “medically significant”
Requires inpatient hospitalization or prolongation of existing hospitalization, excluding the following: 1) routine treatment or monitoring of the underlying disease, not associated with any deterioration in condition; 2) elective or pre-planned treatment for a pre-existing condition that is unrelated to the indication under study and has not worsened since signing the informed consent; and 3) social reasons and respite care in the absence of any deterioration in the patient's general condition.

The terms “once about every week,” “once about every two weeks,” or any other similar dosing interval terms as used herein mean approximate numbers. “Once about every week” can include every seven days ±one day, i.e., every six days to every eight days. “Once about every two weeks” can include every fourteen days ±two days, i.e., every twelve days to every sixteen days. “Once about every three weeks” can include every twenty-one days ±three days, i.e., every eighteen days to every twenty-four days. Similar approximations apply, for example, to once about every four weeks, once about every five weeks, once about every six weeks, and once about every twelve weeks. In some embodiments, a dosing interval of once about every six weeks or once about every twelve weeks means that the first dose can be administered any day in the first week, and then the next dose can be administered any day in the sixth or twelfth week, respectively. In other embodiments, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

As described herein, any concentration range, percentage range, ratio range, or integer range is to be understood to include the value of any integer within the recited range and, when appropriate, fractions thereof (such as one tenth and one hundredth of an integer), unless otherwise indicated.

Various aspects of the disclosure are described in further detail in the following subsections.

### II. Description of the Embodiments

A. Methods for Treating Cancer with Tucatinib

In one aspect, the present invention provides a method for treating cancer in a subject comprising administering a therapeutically effective amount of tucatinib, or salt or solvate thereof, to the subject, wherein the cancer has been determined to express a mutant form of HER2. In some embodiments, the method further comprises determining if the cancer expresses a mutant form of HER2. In one aspect, the present invention provides a method for treating cancer in a subject with a HER2 mutation comprising administering a therapeutically effective amount of tucatinib, or salt or solvate thereof, to the subject. In one aspect, the present invention provides a method for treating cancer in a subject comprising administering a therapeutically effective amount of tucatinib, or salt or solvate thereof, to the subject, wherein the cancer comprises a HER2 mutation. In one aspect the present invention provides a method of inhibiting the kinase activity of HER2 mutants. In some embodiments, the mutant form of HER2 is determined by DNA sequencing. In some embodiments mutant form of HER2 is determined RNA sequencing. In some embodiments, the mutant form of HER2 is determined by nucleic acid sequencing. In some embodiments, the nucleic acid sequencing is next-generation sequencing (NGS). In some embodiments, the mutant form of HER2 is determined by polymerase chain reaction (PCR). In some embodiments, the mutant form of HER2 is determined by analyzing a sample obtained from the subject. In some embodiments, the sample obtained from the subject is a cell-free plasma sample. In some embodiments, the sample obtained from the subject is a tumor biopsy. In some embodiments, the cancer has HER2 amplification. In some embodiments, the cancer does not have HER2 amplification. In some embodiments, the cancer has been determined to comprise a HER2 amplification. In some embodiments, the cancer has been determined to not comprise a HER2 amplification. In some embodiments, HER2 amplification is determined by IHC. In some embodiments, the cancer has a HER2 amplification score of 0, wherein the HER2 amplification score is determined by IHC. In some embodiments, the cancer has a HER2 amplification score of 1+, wherein the HER2 amplification score is determined by IHC. In some embodiments, the cancer has a HER2 amplification score of 0 or 1+, wherein the HER2 amplification score is determined by IHC. In some embodiments, the cancer has a HER2 amplification score of 2+, wherein the HER2 amplification score is determined by IHC. In some embodiments, the cancer has a HER2 amplification score of 3+, wherein the HER2 amplification score is determined by IHC. In some embodiments, HER2 is not amplified if the cancer has a score of 0 as determined by IHC. In some embodiments, HER2 is not amplified if the cancer has a score of 1+ as determined by IHC. In some embodiments, HER2 is amplified if the cancer has a score of 2+ as determined by IHC. In some embodiments, HER2 is amplified if the cancer has a score of 3+ as determined by IHC. In some embodiments, HER2 is amplified if it is overexpressed in the cancer by at least about 5%, about 10%, about 15%, about 20%, about 25%, about 30%, about 35%, about 40%, about 45%, about 50%, about 55%, about 60%, about 70%, about 75%, about 80%, about 85%, about 90%, about 95%, about 100%, about 125%, about 150%, about 175%, about 200%, about 250%, about 300%, about 350%, about 400%, about 450%, or about 500%. In some embodiments, HER2 is amplified if it is overexpressed in the cancer by at least 50%. In some embodiments, HER2 is amplified if it is overexpressed in the cancer by at least 75%. In some embodiments, HER2 is amplified if it is overexpressed in the cancer by at least 100%. In some embodiments, HER2 is amplified if it is overexpressed in the cancer by at least 150%. In some embodiments, HER2 is amplified if it is overexpressed in the cancer by at least 200%. In some embodiments, HER2 is amplified if it is overexpressed in the cancer by at least 250%. In some embodiments, HER2 is amplified if it is overexpressed in the cancer by at least 300%. In some embodiments, HER2 is amplified if it is overexpressed in the cancer by at least 400%. In some embodiments, HER2 is amplified if it is overexpressed in the cancer by at least 500%. In some embodiments, HER2 is amplified if there is at least about a 1.5 fold, about a 2 fold, about a 3 fold, about a 4 fold, about a 5 fold, about a 10 fold, about a 15 fold, about a 20 fold, about a 25 fold, about a 30 fold, about a 40 fold, about a 50 fold, about a 60 fold, about a 70 fold, about a 80 fold, about a 90 fold, or about a 100 fold increase in HER2 protein levels in the cancer. In some embodiments, HER2 is amplified if there is at least about a 1.5 fold increase in HER2 protein levels in the cancer. In some embodiments, HER2 is amplified if there is at least about a 2 fold increase in HER2 protein levels in the cancer. In some embodiments, HER2 is amplified if there is at least about a 3 fold increase in HER2 protein levels in the cancer. In some embodiments, HER2 is amplified if there is at least about a 4 fold increase in HER2 protein levels in the cancer. In some embodiments, HER2 is amplified if there is at least about a 5 fold increase in HER2 protein levels in the cancer. In some embodiments, HER2 is amplified if there is at least about a 10 fold increase in HER2 protein levels in the cancer. In some embodiments, HER2 is amplified if there is at least about a 15 fold increase in HER2 protein levels in the cancer. In some embodiments, HER2 is amplified if there is at least about a 20 fold increase in HER2 protein levels in the cancer. In some embodiments, HER2 is amplified if there is at least about a 25 fold increase in HER2 protein levels in the cancer. In some embodiments, HER2 is amplified if there is at least about a 30 fold increase in HER2 protein levels in the cancer. In some embodiments, HER2 is amplified if there is at least about a 40 fold increase in HER2 protein levels in the cancer. In some embodiments, HER2 is amplified if there is at least about a 50 fold increase in HER2 protein levels. In some embodiments, HER2 is amplified if there is at least about a 60 fold increase in HER2 protein levels in the cancer. In some embodiments, HER2 is amplified if there is at least about a 70 fold increase in HER2 protein levels in the cancer. In some embodiments, HER2 is amplified if there is at least about an 80 fold increase in HER2 protein levels in the cancer. In some embodiments, HER2 is amplified if there is at least about a 90 fold increase in HER2 protein levels in the cancer. In some embodiments, HER2 is amplified if there is at least about a 100 fold increase in HER2 protein levels in the cancer. In some embodiments, the mutant form of HER2 comprises at least one amino acid substitution, insertion, or deletion compared to the human wild-type HER2 amino acid sequence. In some embodiments, wild-type HER2 comprises the amino acid sequence of MELAALCRWGLLLALLPPGAASTQVCTGTDMKLRLPASPETHLDMLRHLYQGCQVVQ GNLELTYLPTNASLSFLQDIQEVQGYVLIAHNQVRQVPLQRLRIVRGTQLFEDNYALAV LDNGDPLNNTTPVTGASPGGLRELQLRSLTEILKGGVLIQRNPQLCYQDTILWKDIFHKN NQLALTLIDTNRSRACHPCSPMCKGSRCWGES SEDCQSLTRTVCAGGCARCKGPLPTDC CHEQCAAGCTGPKHSDCLACLHFNHSGICELHCPALVTYNTDTFESMPNPEGRYTFGAS CVTACPYNYLSTDVGSCTLVCPLHNQEVTAEDGTQRCEKCSKPCARVCYGLGMEHLRE VRAVTSANIQEFAGCKKIFGSLAFLPESFDGDPASNTAPLQPEQLQVFETLEEITGYLYIS AWPDSLPDLSVFQNLQVIRGRILHNGAYSLTLQGLGISWLGLRSLRELGSGLALIEIHNTH LCFVHTVPWDQLFRNPHQALLHTANRPEDECVGEGLACHQLCARGHCWGPGPTQCVN CSQFLRGQECVEECRVLQGLPREYVNARHCLPCHPECQPQNGSVTCFGPEADQCVACA HYKDPPFCVARCPSGVKPDLSYMPIWKFPDEEGACQPCPINCTHSCVDLDDKGCPAEQR ASPLTSIISAVVGILLVVVLGVVFGILIKRRQQKIRKYTMRRLLQETELVEPLTPSGAMPN QAQMRILKETELRKVKVLGSGAFGTVYKGIWIPDGENVKIPVAIKVLRENTSPKANKEIL DEAYVMAGVGSPYVSRLLGICLTSTVQLVTQLMPYGCLLDHVRENRGRLGSQDLLNVVC MQIAKGMSYLEDVRLVEIRDLAARNVLVKSPNHVKITDFGLARLLDIDEIEYHADGGKV PIKWMALESILRRRFTHQSDVWSYGVTVWELMTFGAKPYDGIPAREIPDLLEKGERLPQ PPICTIDVYMIMVKCWMIDSECRPRFRELVSEFSRMARDPQRFVVIQNEDLGPASPLDST FYRSLLEDDDMGDLVDAEEYLVPQQGFFCPDPAPGAGGMVEIHREIRSSSTRSGGGDLTL GLEPSEEEAPRSPLAPSEGAGSDVFDGDLGMGAAKGLQSLPTHDPSPLQRYSEDPTVPLP SETDGYVAPLTCSPQPEYVNQPDVRPQPPSPREGPLPAARPAGATLERPKTLSPGKNGVV KDVFAFGGAVENPEYLTPQGGAAPQPHPPPAFSPAFDNLYYWDQDPPERGAPPSTFKGT PTAENPEYLGLDVPV (SEQ ID NO:1). In some embodiments, the mutation in HER2 is an activating mutation. In some embodiments, the mutation is in the extracellular domain of HER2. In some embodiments, the mutation is in the transmembrane domain of HER2. In some embodiments, the mutation is in the juxtamembrane domain of HER2. In some embodiments, the mutation is in the kinase domain of HER2. In some embodiments, the mutant form of HER2 comprises the amino acid substitution L755S. In some embodiments, the mutant form of HER2 comprises the amino acid substitution V777L. In some embodiments, the mutant form of HER2 comprises the amino acid substitution S310Y. In some embodiments, the mutant form of HER2 comprises a G776 YVMA insertion (G776 ins YVMA). The G776 ins YVMA mutant form of HER2 is a mutant in which YVMA (SEQ ID NO: 2) (tyrosine, valine, methionine, alanine), which is the amino acid sequence at positions 772 to 775 of the HER2 protein, is repeated once again (also referred to as “Y772_A775dup” or “A775_G776insYVMA”). Nature. 2004 Sep. 30; 431 (7008): 525-6, and Cancer Res. 2005 Mar. 1; 65 (5): 1642-6. In some embodiments, the HER2 mutation results in constitutive HER2 kinase domain activation. In some embodiments, the cancer is selected from the group consisting of gastric cancer, colorectal cancer, lung cancer, gall bladder cancer, and breast cancer. In some embodiments, the cancer is gastric cancer. In some embodiments, the cancer is colorectal cancer. In some embodiments, the cancer is lung cancer. In some embodiments, the lung cancer is non-small cell lung cancer. In some embodiments the cancer is gall bladder cancer. In some embodiments, the cancer is breast cancer. In some embodiments, the breast cancer is HER2 positive breast cancer. In some embodiments, the cancer is gastric cancer and comprises an activating HER2 mutation. In some embodiments, the cancer is gastric cancer and comprises a mutant form of HER2 comprising the amino acid substitution L755S. In some embodiments, the cancer is gastric cancer and comprises a mutant form of HER2 comprising the amino acid substitution V777L. In some embodiments, the cancer is gastric cancer and comprises a mutant form of HER2 comprising the amino acid substitution S310Y. In some embodiments, the cancer is gastric cancer and comprises a mutant form of HER2 comprising a G776 YVMA insertion (G776 ins YVMA). In some embodiments, the cancer is colorectal cancer and comprises an activating HER2 mutation. In some embodiments, the cancer is colorectal cancer and comprises a mutant form of HER2 comprising the amino acid substitution L755S. In some embodiments, the cancer is colorectal cancer and comprises a mutant form of HER2 comprising the amino acid substitution V777L. In some embodiments, the cancer is colorectal cancer and comprises a mutant form of HER2 comprising the amino acid substitution S310Y. In some embodiments, the cancer is colorectal cancer and comprises a mutant form of HER2 comprising a G776 YVMA insertion (G776 ins YVMA). In some embodiments, the cancer is lung cancer, such as non-small cell lung cancer, and comprises an activating HER2 mutation. In some embodiments, the cancer is lung cancer, such as non-small cell lung cancer, and comprises a mutant form of HER2 comprising the amino acid substitution L755S. In some embodiments, the cancer is lung cancer, such as non-small cell lung cancer, and comprises a mutant form of HER2 comprising the amino acid substitution V777L. In some embodiments, the cancer is lung cancer, such as non-small cell lung cancer, and comprises a mutant form of HER2 comprising the amino acid substitution S310Y. In some embodiments, the cancer is lung cancer, such as non-small cell lung cancer, and comprises a mutant form of HER2 comprising a G776 YVMA insertion (G776 ins YVMA). In some embodiments, the cancer is gall bladder cancer and comprises an activating HER2 mutation. In some embodiments, the cancer is gall bladder cancer and comprises a mutant form of HER2 comprising the amino acid substitution L755S. In some embodiments, the cancer is gall bladder cancer and comprises a mutant form of HER2 comprising the amino acid substitution V777L. In some embodiments, the cancer is gall bladder cancer and comprises a mutant form of HER2 comprising the amino acid substitution S310Y. In some embodiments, the cancer is gall bladder cancer and comprises a mutant form of HER2 comprising a G776 YVMA insertion (G776 ins YVMA). In some embodiments, the cancer is breast cancer, such as HER2 positive breast cancer, and comprises an activating HER2 mutation. In some embodiments, the cancer is breast cancer, such as HER2 positive breast cancer, and comprises a mutant form of HER2 comprising the amino acid substitution L755S. In some embodiments, the cancer is breast cancer, such as HER2 positive breast cancer, and comprises a mutant form of HER2 comprising the amino acid substitution V777L. In some embodiments, the cancer is breast cancer, such as HER2 positive breast cancer, and comprises a mutant form of HER2 comprising the amino acid substitution S310Y. In some embodiments, the cancer is breast cancer, such as HER2 positive breast cancer, and comprises a mutant form of HER2 comprising a G776 YVMA insertion (G776 ins YVMA).

In some embodiments, the cancer is metastatic. In some embodiments, the cancer has metastasized to the brain. In some embodiments, the cancer is locally advanced. In some embodiments, the cancer is unresectable. In some embodiments, the subject has been previously treated with one or more additional therapeutic agents for the cancer. In some embodiments, the subject has been previously treated with one or more additional therapeutic agents for the cancer and did not respond to the treatment. In some embodiments, the subject has been previously treated with one or more additional therapeutic agents for the cancer and relapsed after the treatment. In some embodiments, the subject has been previously treated with one or more additional therapeutic agents for the cancer and experienced disease progression during the treatment. In some embodiments, the one or more additional therapeutic agents is an anti-HER2 antibody or anti-HER2 antibody-drug conjugate. In some embodiments, the one or more additional therapeutic agents is an anti-HER2 antibody. In some embodiments, the one or more additional therapeutic agents is anti-HER2 antibody-drug conjugate. In some embodiments, the subject has been previously treated with trastuzumab, pertuzumab and/or T-DM1. In some embodiments, the subject has been previously treated with trastuzumab. In some embodiments, the subject has been previously treated with pertuzumab. In some embodiments, the subject has been previously treated with T-DM1. In some embodiments, the subject has been previously treated with trastuzumab and pertuzumab. In some embodiments, the subject has been previously treated with trastuzumab and T-DM1. In some embodiments, the subject has been previously treated with pertuzumab and T-DM1. In some embodiments, the subject has been previously treated with trastuzumab, pertuzumab and T-DM1. In some embodiments, the subject has not been previously treated with another therapeutic agent for the cancer within the past 1 day, 2 days, 3 days, 4 days, 5 days, 6 days, 7 days, 10 days, 2 weeks, 3 weeks, 4 weeks, 6 weeks, 2 months, 3 months, 7 months, 8 months, 9 months, 10 months, 11 months, 12 months, 15 months, 18 months, 2 years, 3 years, 4 years, 5 years, 6 years, 7 years, 8 years, 9 years or 10 years prior to being administered the therapeutically effective amount of tucatinib, or salt or solvate thereof. In some embodiments, the subject has not been previously treated with another therapeutic agent for the cancer within the past 12 months prior to being administered the therapeutically effective amount of tucatinib, or salt or solvate thereof. In some embodiments, the subject has not been previously treated with another therapeutic agent for the cancer. In some embodiments, the subject has not been previously treated with lapatinib, neratinib, afatinib, or capecitabine. In some embodiments, the subject has not been previously treated with lapatinib. In some embodiments, the subject has not been previously treated with neratinib. In some embodiments, the subject has not been previously treated with afatinib. In some embodiments, the subject has not been previously treated with capecitabine.

In some embodiments, ther HER2 status of a sample cell is determined. The determination can be made before treatment (i.e., administration of tucatinib) begins, during treatment, or after treatment has been completed. In some instances, determination of the HER2 status results in a decision to change therapy (e.g., adding an anti-HER2 antibody to the treatment regimen, discontinuing the use of tucatinib, discontinuing therapy altogether, or switching from another treatment method to a method of the present invention).

In some embodiments, the sample cell is a cancer cell. In some instances, the sample cell is obtained from a subject who has cancer. The sample cell can be obtained as a biopsy specimen, by surgical resection, or as a fine needle aspirate (FNA). In some embodiments, the sample cell is a circulating tumor cell (CTC).

HER2 expression can be compared to a reference cell. In some embodiments, the reference cell is a non-cancer cell obtained from the same subject as the sample cell. In other embodiments, the reference cell is a non-cancer cell obtained from a different subject or a population of subjects. In some embodiments, measuring expression of HER2 comprises, for example, determining HER2 gene copy number or amplification, nucleic acid sequencing (e.g., sequencing of genomic DNA or cDNA or RNA sequencing), measuring mRNA expression, measuring protein abundance, or a combination thereof. HER2 testing methods include immunohistochemistry (IHC), in situ hybridization, fluorescence in situ hybridization (FISH), chromogenic in situ hybridization (CISH), ELISAs, and RNA quantification (e.g., of HER2 expression) using techniques such as RT-PCR and microarray analysis.

In some embodiments, the presence or absence of a HER2 mutation is confirmed by, for example, collecting tumor tissue from a cancer patient and performing a method such as real-time quantitative PCR (qRT-PCR) or microarray analysis. In some embodiments, the tumor tissue is a formalin-fixed paraffin-embedded specimen (FFPE). In some embodiments, the presence or absence of HER2 mutation is confirmed by collecting acellular circulating tumor DNA (ctDNA) from a cancer patient and performing a method such as next generation sequencing (NGS) (J Clin Oncol 2013; 31: 1997-2003, Clin Cancer Res 2012; 18: 4910-8, J Thorac Oncol 2012; 7: 85-9, Lung Cancer 2011; 74: 139-44, Cancer Res 2005; 65: 1642-6, Cancer Sci 2006; 97: 753-9, and ESMO Open 2017; 2: e000279).

Nucleic acids used to detect HER2 mutations in any of the methods described herein include genomic DNA, RNA transcribed from genomic DNA, and cDNA generated from RNA. Nucleic acids can be derived from vertebrates, for example mammals. A nucleic acid is said to be directly derived from a particular source or “derived from” a particular source if it is a copy of a nucleic acid found in that source.

In certain embodiments, the nucleic acid comprises a copy of the nucleic acid, e.g., a copy resulting from amplification. For example, amplification to obtain the desired amount of material to detect mutations may be desirable in certain instances. The amplicon may then go through a mutation detection method, such as those described below, to determine whether the mutation is present in the amplicon.

Somatic mutations or variations can be detected by certain methods known to those skilled in the art. Such methods include, but are not limited to, DNA sequencing, primers including somatic mutation-specific nucleotide incorporation assays and somatic mutation-specific primer extension assays (e.g., somatic mutation-specific PCR, somatic mutation-specific ligation chain reaction (LCR), and gap-LCR extension assays), mutation-specific oligonucleotide hybridization assays (e.g., oligonucleotide ligation assays), cleavage protection assays in which protection from cleavage agents is used to detect fluorinated bases in nucleic acid duplexes, electrophoretic analysis comparing the mobility of variants and wild type nucleic acid molecules, denaturation-gradient gel electrophoresis (e.g., DGGE as in Myers et al. (1985) Nature 313: 495), analysis of RNase cleavage on uninched base pairs, analysis of chemical or enzymatic cleavage of heteroduplex DNA, mass spectrometry (e.g., MALDI-TOF); genetic bit analysis (GBA), 5′nuclease assay (e.g., TaqMan™), and assays using molecular pathway labels.

Detection of variation in the target nucleic acid can be accomplished by molecular cloning and sequencing of the target nucleic acid using techniques well known in the art. Alternatively, amplification techniques such as polymerase chain reaction (PCR) can be used to amplify target nucleic acid sequences directly from genomic DNA preparations from tumor tissue. The nucleic acid sequence of the amplified sequence can then be determined and variations identified therefrom. Amplification techniques are well known in the art, for example, polymerase chain reactions are described in Saiki et al., Science 239: 487, 1988; U.S. Pat. Nos. 4,683,203 and 4,683,195.

Ligase chain reactions known in the art can also be used to amplify target nucleic acid sequences. See, e.g., Wu et al., Genomics 4: 560-569 (1989). Also, a technique known as allele-specific PCR can also be used to detect somatic mutations (e.g., substitutions). See, e.g., Ruano and Kidd (1989) Nucleic Acids Research 17: 8392; McClay et al. (2002) Analytical Biochem. 301: 200-206. In certain embodiments of this technique, the 3′terminal nucleotides of the primers are complementary to (i.e., specifically form base pairs with) certain variations of the target nucleic acid. Mutation-specific primers are used. If no specific mutation is present, no amplification product is observed. Amplification resistance mutation systems (ARMS) can also be used to detect variations (e.g., substitutions). ARMS is described, for example, in European Patent Application Publication No. 0332435, and Newton et al., Nucleic Acids Research, 17: 7, 1989.

Other methods useful for detecting variations (e.g., substitutions) include, but are not limited to: (1) mutation-specific nucleotide incorporation assays, such as single base extension assays (see, e.g., Chen et al. (2000) Genome Res. 10: 549-557); (2) mutation-specific primer extension assays (see, e.g., Ye et al. (2001) Hum. Mut. 17: 305-316); (3) 5′nuclease assay (see, e.g., De La Vega et al. (2002) BioTechniques 32: S48-S54 (which describes the TaqMan® assay); (4) assays using molecular pathway labels (see, e.g., Tyagi et al. (1998) Nature Biotech. 16: 49-53); (5) oligonucleotide ligation assays (see, e.g., Grossman et al. (1994) Nuc. Acids Res. 22: 4527-4534) and (6) allele-specific PCR;

Variations can also be detected by mismatch detection methods. Mismatches are hybridized nucleic acid duplexes that are not 100% complementary. Lack of total complementarity can be attributed to deletions, insertions, inversions, or substitutions. One example of a mismatch detection method is, for example, a mismatch recovery detection (MRD) assay described in Faham et al., Proc. Natl. Acad. Sci. USA 102: 14717-14722 (2005). Another example of a mismatched cutting technique is the RNase protection method described in detail in Myers et al., Science 230: 1242, 1985. For example, the methods used to detect variation may include the use of labeled riboprobes that are complementary to human wild type target nucleic acids. Riboprobes and target nucleic acids derived from tissue samples are annealed (hybridized) together and subsequently digested with the enzyme RNase A, which can detect some mismatches in the duplex RNA structure. If a mismatch is detected by RNase A, it is cleaved at the site of the mismatch. Thus, when annealed RNA preparations are separated on an electrophoretic gel matrix, if mismatches are detected and cleaved by RNase A, smaller RNA products will be observed than mRNA or full length duplex RNA for DNA and riboprobes. Riboprobes need not be the full length of the target nucleic acid, but can be part of the target nucleic acid, as long as it includes a position suspected of having a mutation.

In a similar manner, DNA probes can be used to detect mismatches, for example, via enzymatic or chemical cleavage. For example, Cotton et al., Proc. Natl. Acad. Sci. USA, 85: 4397, 1988. Alternatively, discrepancies can be detected by the transition of the electrophoretic mobility of the mismatched duplex to the matched duplex. See, e.g., Cariello, Human Genetics, 42: 726, 1988. With either riboprobes or DNA probes, target nucleic acids suspected of containing mutations can be amplified prior to hybridization. In particular, if the change is a severe rearrangement such as deletion and insertion, changes in the target nucleic acid can also be detected using Southern hybridization.

Restriction fragment length polymorphism (RFLP) probes to target nucleic acids or surrounding marker genes can be used to detect variations, for example insertions or deletions. Insertions and deletions can also be detected by cloning, sequencing and amplification of target nucleic acids. Single stranded polymorphism (SSCP) assays can also be used to detect base altering variants of the allele. SSCP can be modified for the detection of ErbB2 somatic mutations. SSCP identifies base differences due to alterations in electrophoretic shifting of single stranded PCR products. Single-stranded PCR products can be produced by heating or otherwise denaturing the double-stranded PCR product. Single-stranded nucleic acids may refold or form secondary structures that are partially dependent on the base sequence. Different electrophoretic mobility of single-stranded amplification products is related to base-sequence differences at SNP positions. Denaturation gradient gel electrophoresis (DGGE) differentiates SNP alleles based on different sequence-dependent stability and melting characteristics inherent to polymorphic DNA and corresponding differences in electrophoretic migration patterns in denaturing gradient gels.

Somatic mutations or modifications can also be detected using microarrays. Microarrays are typically a multiplex technique using a series of thousands of nucleic acid probes arranged to hybridize under high-stringency conditions, e.g., with a cDNA or cRNA sample. Probe-target hybridization is typically detected and quantified by detection of fluorophore-, silver-, or chemiluminescent-labeled targets to determine the relative abundance of nucleic acid sequences at the target. In a typical microarray, the probe is attached to a hard surface by covalent bonds to the chemical matrix (via epoxy-silane, amino-silane, lysine, polyacrylamide or the like). Hard surfaces are, for example, glass, silicon chips, or microscopic beads.

Another method for the detection of somatic mutations is based on mass spectrometry. Mass spectrometry uses the unique mass of each of the four nucleotides of DNA. Potential mutation-containing ErbB2 nucleic acids can be clearly analyzed by mass spectrometry by measuring the difference in mass of nucleic acids with somatic mutations. MALDI-TOF (matrix assisted laser desorption ionization-timeout) mass spectrometry techniques are useful for extremely accurate determination of molecular weight, such as nucleic acids containing somatic mutations. Numerous approaches to nucleic acid analysis have been developed based on mass spectrometry. Exemplary mass spectrometry-based methods also include primer extension assays, which can be used in combination with other approaches, such as traditional gel-based formats and microarrays.

Sequence-specific ribozymes (U.S. Pat. No. 5,498,531) can also be used to detect somatic mutations based on the development or loss of ribozyme cleavage sites. Perfectly matched sequences can be distinguished from mismatched sequences by nuclease cleavage digestion assays or differences in melting temperatures. If a mutation affects a restriction enzyme cleavage site, the mutation can be identified by a change in the restriction enzyme digestion pattern and a corresponding change in nucleic acid fragment length determined by gel electrophoresis.

In certain embodiments of the present disclosure, protein-based detection techniques are used to detect variant proteins encoded by genes with genetic variations as disclosed herein. Determination of the presence of variant forms of proteins can be performed by any suitable technique known in the art, for example electrophoresis (e.g., denatured or non-modified polyacrylamide gel electrophoresis, two-dimensional gel electrophoresis, capillary electrophoresis). Electrophoresis, and isoelectronic focusing, chromatography (e.g., sizing chromatography, high performance liquid chromatography (HPLC), and cation exchange HPLC), mass spectroscopy (e.g., MALDI-TOF mass spectroscopy, electrospray), ionization (ESI) mass spectroscopy, and tandem mass spectroscopy). See, e.g., Ahrer and Jungabauer (2006) J. Chromatog. B. Analyt. Technol. Biomed. Life Sci. 841: 110-122. A suitable technique can be selected based in part on the nature of the variation detected. For example, variations in which substituted amino acids result in amino acid substitutions with charges different from the original amino acids can be detected by isoelectric point electrophoresis. Isoelectric electrophoresis of a polypeptide through a gel with a pH gradient at high voltage separates the protein by its isoelectric point (pi). pH gradient gels can be compared to co-operated gels containing wild type protein. In instances where the mutation results in the generation of new proteolytic cleavage sites or the abolition of existing ones, the samples can be peptide mapped using proteolytic digestion followed by appropriate electrophoresis, chromatography, or mass spectrometry techniques. The presence of the variation can also be detected using protein sequencing techniques such as Edman degradation or certain forms of mass spectroscopy.

Methods known in the art using a combination of these techniques can also be used. For example, in HPLC-microscopy tandem mass spectrometry techniques, proteolytic digestion is performed on proteins and the resulting peptide mixtures are separated by reverse phase chromatography separation. Tandem mass spectrometry is then performed and the data collected therefrom are analyzed. In another example, unmodified gel electrophoresis is combined with MALDI mass spectroscopy

In certain embodiments, a protein can be isolated from a sample using reagents such as antibodies or peptides that specifically bind to the protein, and then further analyzed to present the genetic variation using any of the techniques disclosed above.

Alternatively, the presence of the variant protein in the sample may be directed to an antibody specific for a protein having a genetic variation, i.e., an antibody that specifically binds to a protein having a mutation but does not bind to a protein having no mutation. It can be detected by an immunoaffinity assay. Such antibodies can be produced by any suitable technique known in the art. Antibodies can be used to immunoprecipitate a particular protein from a solution sample or to immunoblot a protein separated by, for example, a polyacrylamide gel. Immunocytochemical methods can also be used to detect specific protein variants in tissues or cells. For example, immunoenzymatic assays (IEMA), including enzyme-linked immunosorbent assays (ELISA), radioimmunoassay (RIA), immunoradiometric (IRMA) and sandwich assays using monoclonal or polyclonal antibodies.

B. Tucatinib Dose and Administration

In some embodiments, a dose of tucatinib is between about 0.1 mg and 10 mg per kg of the subject's body weight (e.g., about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 mg per kg of the subject's body weight). In other embodiments, a dose of tucatinib is between about 10 mg and 100 mg per kg of the subject's body weight (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 mg per kg of the subject's body weight). In some embodiments, a dose of tucatinib is at least about 100 mg to 500 mg per kg of the subject's body weight (e.g., at least about 100, 125, 150, 175, 200, 225, 250, 275, 300, 325, 350, 375, 400, 425, 450, 475, or 500 mg per kg of the subject's body weight). In particular embodiments, a dose of tucatinib is between about 1 mg and 50 mg per kg of the subject's body weight (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg per kg of the subject's body weight). In some instances, a dose of tucatinib is about 50 mg per kg of the subject's body weight.

In some embodiments, a dose of tucatinib comprises between about 1 mg and 100 mg (e.g. about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 mg) of tucatinib. In other embodiments, a dose of tucatinib comprises between about 100 mg and 1,000 mg (e.g., about 100, 105, 110, 115, 120, 125, 130, 135, 140, 145, 150, 155, 160, 165, 170, 175, 180, 185, 190, 195, 200, 205, 210, 215, 220, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1,000 mg) of tucatinib. In particular embodiments, a dose of tucatinib is about 300 mg (e.g., when administered twice per day).

In some embodiments, a dose of tucatinib comprises at least about 1,000 mg to 10,000 mg (e.g., at least about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000 or more mg) of tucatinib.

In some embodiments, a dose of tucatinib, or salt or solvate thereof, contains a therapeutically effective amount of tucatinib, or salt or solvate thereof. In other embodiments, a dose of tucatinib, or salt or solvate thereof, contains less than a therapeutically effective amount of tucatinib, or salt or solvate thereof, (e.g., when multiple doses are given in order to achieve the desired clinical or therapeutic effect).

Tucatinib, or salt or solvate thereof, can be administered by any suitable route and mode. Suitable routes of administering antibodies and/or antibody-drug conjugate of the present invention are well known in the art and may be selected by those of ordinary skill in the art. In one embodiment, tucatinib administered parenterally. Parenteral administration refers to modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion. In some embodiments, the route of administration of tucatinib is intravenous injection or infusion. In some embodiments, the route of administration of tucatinib is intravenous infusion. In some embodiments, the route of administration of tucatinib is intravenous injection or infusion. In some embodiments, the tucatinib is intravenous infusion. In some embodiments, the route of administration of tucatinib is oral.

In one embodiment of the methods or uses or product for uses provided herein, tucatinib is administered to the subject daily, twice daily, three times daily or four times daily. In some embodiments, tucatinib is administered to the subject every other day, once about every week or once about every three weeks. In some embodiments, tucatinib is administered to the subject once per day. In some embodiments, tucatinib is administered to the subject twice per day. In some embodiments, tucatinib is administered to the subject at a dose of about 300 mg twice per day. In some embodiments, tucatinib is administered to the subject at a dose of 300 mg twice per day. In some embodiments, tucatinib is administered to the subject at a dose of about 600 mg once per day. In some embodiments, tucatinib is administered to the subject at a dose of 600 mg once per day. In some embodiments, tucatinib is administered to the subject twice per day on each day of a 21 day treatment cycle. In some embodiments, the tucatinib is administered to the subject orally.

C. Combination Therapy

In some aspects, a method of treatment as described herein further comprises administering one or more additional therapeutic agents to the subject to treat the cancer. In some embodiments, the one or more additional therapeutic agents is selected from the group consisting of capecitabine and an anti-HER2 antibody. In some embodiments, the one or more additional therapeutic agents is capecitabine. In some embodiments, the one or more additional therapeutic agents is an anti-HER2 antibody. In some embodiments, the one or more additional therapeutic agents are capecitabine and an anti-HER2 antibody. In some embodiments, the anti-HER2 antibody is selected from the group consisting of trastuzumab, pertuzumab, ado-trastuzumab emtansine, margetuximab, and a combination thereof. In some instances, the anti-HER2 antibody is a combination of trastuzumab and pertuzumab. In some embodiments, the anti-HER2 antibody is trastuzumab. In some embodiments, the one or more additional therapeutic agents are capecitabine and trasuzumab.

In some embodiments, a method of treatment described herein further comprises administering capecitabine to the subject at a dose based on the body surface area of the subject. In some embodiments, capecitabine is administered to the subject at a dose of about 500 mg/m² to about 1500 mg/m². In some embodiments, capecitabine is administered to the subject at a dose of about 500 mg/m², about 550 mg/m², about 600 mg/m², about 650 mg/m², about 700 mg/m², about 750 mg/m², about 800 mg/m², about 850 mg/m², about 900 mg/m², about 950 mg/m², about 1000 mg/m², about 1050 mg/m², about 1100 mg/m², about 1150 mg/m², about 1200 mg/m², about 1250 mg/m², about 1300 mg/m², about 1350 mg/m², about 1400 mg/m², about 1450 mg/m², or about 1500 mg/m². In some embodiments, capecitabine is administered to the subject at a dose of 500 mg/m² to 1500 mg/m². In some embodiments, capecitabine is administered to the subject at a dose of 500 mg/m², 550 mg/m², 600 mg/m², 650 mg/m², 700 mg/m², 750 mg/m², 800 mg/m², 850 mg/m², 900 mg/m², 950 mg/m², 1000 mg/m², 1050 mg/m², 1100 mg/m², 1150 mg/m², 1200 mg/m², 1250 mg/m², 1300 mg/m², 1350 mg/m², 1400 mg/m², 1450 mg/m², or 1500 mg/m². In some embodiments, capecitabine is administered to the subject daily, twice daily, three times daily or four times daily. In some embodiments, capecitabine is administered to the subject every other day, once about every week or once about every three weeks. In some embodiments, capecitabine is administered to the subject once per day. In some embodiments, capecitabine is administered to the subject twice per day. In some embodiments, capecitabine is administered to the subject twice per day on days 1-14 of a 21 day treatment cycle. In some embodiments, capecitabine is administered to the subject at a dose of about 1000 mg/m² twice per day. In some embodiments, capecitabine is administered to the subject at a dose of 1000 mg/m² twice per day. In some embodiments, capecitabine is administered to the subject at a dose of about 1000 mg/m² twice per day on days 1-14 of a 21 day treatment cycle. In some embodiments, capecitabine is administered to the subject at a dose of 1000 mg/m² twice per day on days 1-14 of a 21 day treatment cycle. In some embodiments, the capecitabine is administered to the subject orally.

In some embodiments, a method of treatment described herein further comprises administering an anti-HER2 antibody to the subject. In some embodiments, a dose of the anti-HER2 antibody is between about 0.1 mg and 10 mg per kg of the subject's body weight (e.g., about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 mg per kg of the subject's body weight). In some embodiments, a dose of the anti-HER2 antibody is between about 4 mg and 10 mg per kg of the subject's body weight. In some embodiments, a dose of the anti-HER2 antibody is between 4 mg and 10 mg per kg of the subject's body weight. In some embodiments, a dose of the anti-HER2 antibody is about 6 mg per kg of the subject's body weight. In some embodiments, a dose of the anti-HER2 antibody is about 8 mg per kg of the subject's body weight. In some embodiments, a dose of the anti-HER2 antibody is about 8 mg per kg of the subject's body weight for the first dose of the anti-HER2 antibody administered to the subject followed by subsequent doses of about 6 mg per kg of the subject's body weight. In some embodiments, a dose of the anti-HER2 antibody is 6 mg per kg of the subject's body weight. In some embodiments, a dose of the anti-HER2 antibody is 8 mg per kg of the subject's body weight. In some embodiments, a dose of the anti-HER2 antibody is 8 mg per kg of the subject's body weight for the first dose of the anti-HER2 antibody administered to the subject followed by subsequent doses of 6 mg per kg of the subject's body weight. In other embodiments, a dose of the anti-HER2 antibody is between about 10 mg and 100 mg per kg of the subject's body weight (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 mg per kg of the subject's body weight). In some embodiments, a dose of the anti-HER2 antibody is at least about 100 mg to 500 mg per kg of the subject's body weight (e.g., at least about 100, 125, 150, 175, 200, 225, 250, 275, 300, 325, 350, 375, 400, 425, 450, 475, 500, or more mg per kg of the subject's body weight). In some instances, a dose of the anti-HER2 antibody is about 6 mg per kg of the subject's body weight. In other instances, a dose of the anti-HER2 antibody is about 8 mg per kg of the subject's body weight. In some other instances, a dose of the anti-HER2 antibody is about 20 mg per kg of the subject's body weight. In some embodiments, a dose of the anti-HER2 antibody comprises between about 1 mg and 100 mg (e.g. about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 mg) of the anti-HER2 antibody. In other embodiments, a dose of the anti-HER2 antibody comprises between about 100 mg and 1,000 mg (e.g., about 100, 105, 110, 115, 120, 125, 130, 135, 140, 145, 150, 155, 160, 165, 170, 175, 180, 185, 190, 195, 200, 205, 210, 215, 220, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1,000 mg) of the anti-HER2 antibody. In particular embodiments, a dose of the anti-HER2 antibody comprises between about 100 mg and 400 mg (e.g., about 100, 125, 150, 175, 200, 225, 250, 275, 300, 325, 350, 375, or 400 mg) of the anti-HER2 antibody. In some embodiments, a dose of the anti-HER2 antibody is between about 400 mg and 800 mg. In some embodiments, a dose of the anti-HER2 antibody is between 400 mg and 800 mg. In some embodiments, a dose of the anti-HER2 antibody is about 600 mg. In some embodiments, a dose of the anti-HER2 antibody is 600 mg. As a non-limiting example, when using a dose of 6 mg/kg, a dose for a 50 kg subject will be about 300 mg. As another non-limiting example, when using a dose of 8 mg/kg, a dose for a 50 kg subject will be about 400 mg. In some embodiments, a dose of the anti-HER2 antibody comprises at least about 1,000 mg to 10,000 mg (e.g., at least about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000 or more mg) of the anti-HER2 antibody. In some embodiments, a dose of the anti-HER2 antibody contains a therapeutically effective amount of the anti-HER2 antibody. In other embodiments, a dose of the anti-HER2 antibody contains less than a therapeutically effective amount of the anti-HER2 antibody (e.g., when multiple doses are given in order to achieve the desired clinical or therapeutic effect). In some embodiments, the anti-HER2 antibody is administered to the subject once about every 1 to 4 weeks. In certain embodiments, an anti-HER2 antibody is administered once about every 1 week, once about every 2 weeks, once about every 3 weeks or once about every 4 weeks. In one embodiment, an anti-HER2 antibody is administered once about every 3 weeks. In some embodiments, the anti-HER2 antibody is administered to the subject once every 1 to 4 weeks. In certain embodiments, an anti-HER2 antibody is administered once every 1 week, once about every 2 weeks, once about every 3 weeks or once about every 4 weeks. In one embodiment, an anti-HER2 antibody is administered once every 3 weeks. In some embodiments, the anti-HER2 antibody is administered to the subject subcutaneously. In some embodiments, the anti-HER2 antibody is administered to the subject intraperitoneally. In some embodiments, the anti-HER2 antibody is administered to the subject intravenously. In some embodiments, the anti-HER2 antibody is selected from the group consisting of trastuzumab, pertuzumab, ado-trastuzumab emtansine, margetuximab, and a combination thereof. In some instances, the anti-HER2 antibody is a combination of trastuzumab and pertuzumab. In some embodiments, the anti-HER2 antibody is trastuzumab. In some embodiments, the anti-HER2 antibody is administered at a dose of about 600 mg once about every 3 weeks and the anti-HER2 antibody is administered subcutaneously. In some embodiments, the anti-HER2 antibody is administered at a dose of 600 mg once every 3 weeks and the anti-HER2 antibody is administered subcutaneously. In some embodiments, the anti-HER2 antibody is trastuzumab and is administered at a dose of about 600 mg once about every 3 weeks and the trastuzumab is administered subcutaneously. In some embodiments, the anti-HER2 antibody is trastuzumab and is administered at a dose of 600 mg once every 3 weeks and the trastuzumab is administered subcutaneously. In some embodiments, the anti-HER2 antibody is administered at a dose of about 6 mg/kg once about every 3 weeks and the anti-HER2 antibody is administered intravenously. In some embodiments, the anti-HER2 antibody is administered at a dose of about 8 mg/kg once about every 3 weeks and the anti-HER2 antibody is administered intravenously. In some embodiments, the anti-HER2 antibody is administered once about every 3 weeks at a dose of about 8 mg/kg for the first dose of the anti-HER2 antibody administered to the subject followed by subsequent doses of about 6 mg/kg, wherein anti-HER2 antibody is administered intravenously. In some embodiments, the anti-HER2 antibody is administered at a dose of 6 mg/kg once every 3 weeks and the anti-HER2 antibody is administered intravenously. In some embodiments, the anti-HER2 antibody is administered at a dose of 8 mg/kg once every 3 weeks and the anti-HER2 antibody is administered intravenously. In some embodiments, the anti-HER2 antibody is administered once every 3 weeks at a dose of 8 mg/kg for the first dose of the anti-HER2 antibody administered to the subject followed by subsequent doses of 6 mg/kg, wherein anti-HER2 antibody is administered intravenously. In some embodiments, the anti-HER2 antibody is trastuzumab and is administered at a dose of about 6 mg/kg once about every 3 weeks and the trastuzumab is administered intravenously. In some embodiments, the anti-HER2 antibody is trastuzumab and is administered at a dose of about 8 mg/kg once about every 3 weeks and the trastuzumab is administered intravenously. In some embodiments, the anti-HER2 antibody is trastuzumab and is administered once about every 3 weeks at a dose of about 8 mg/kg for the first dose of the trastuzumab administered to the subject followed by subsequent doses of about 6 mg/kg, wherein the trastuzumab is administered intravenously. In some embodiments, the anti-HER2 antibody is trastuzumab and is administered at a dose of 6 mg/kg once every 3 weeks and the trastuzumab is administered intravenously. In some embodiments, the anti-HER2 antibody is trastuzumab and is administered at a dose of 8 mg/kg once every 3 weeks and the trastuzumab is administered intravenously. In some embodiments, the anti-HER2 antibody is trastuzumab and is administered once every 3 weeks at a dose of 8 mg/kg for the first dose of trastuzumab administered to the subject followed by subsequent doses of 6 mg/kg, wherein the trastuzumab is administered intravenously. In some embodiments, the anti-HER2 antibody is trastuzumab and is administered to the subject on a 21 day treatment cycle and is administered to the subject once per treatment cycle.

In some embodiments, a method of treatment described herein comprises administering to the subject tucatinib, capecitabine and trastuzumab. In some embodiments, the tucatinib, capecitabine and trastuzumab are administered to the subject on a 21 day treatment cycle. In some embodiments, tucatinib is administered to the subject at a dose of about 300 mg twice per day. In some embodiments, tucatinib is administered to the subject at a dose of 300 mg twice per day. In some embodiments, tucatinib is administered to the subject at a dose of about 600 mg once per day. In some embodiments, tucatinib is administered to the subject at a dose of 600 mg once per day. In some embodiments, tucatinib is administered to the subject twice per day on each day of a 21 day treatment cycle. In some embodiments, the tucatinib is administered to the subject orally. In some embodiments, capecitabine is administered to the subject twice per day. In some embodiments, capecitabine is administered to the subject twice per day on days 1-14 of a 21 day treatment cycle. In some embodiments, capecitabine is administered to the subject at a dose of about 1000 mg/m² twice per day. In some embodiments, capecitabine is administered to the subject at a dose of 1000 mg/m² twice per day. In some embodiments, capecitabine is administered to the subject at a dose of about 1000 mg/m² twice per day on days 1-14 of a 21 day treatment cycle. In some embodiments, capecitabine is administered to the subject at a dose of 1000 mg/m² twice per day on days 1-14 of a 21 day treatment cycle. In some embodiments, the capecitabine is administered to the subject orally. In some embodiments, the anti-HER2 antibody is administered at a dose of about 6 mg/kg once about every 3 weeks and the anti-HER2 antibody is administered intravenously. In some embodiments, the anti-HER2 antibody is administered at a dose of about 8 mg/kg once about every 3 weeks and the anti-HER2 antibody is administered intravenously. In some embodiments, the anti-HER2 antibody is administered once about every 3 weeks at a dose of about 8 mg/kg for the first dose of the anti-HER2 antibody administered to the subject followed by subsequent doses of about 6 mg/kg, wherein anti-HER2 antibody is administered intravenously. In some embodiments, the anti-HER2 antibody is administered at a dose of 6 mg/kg once every 3 weeks and the anti-HER2 antibody is administered intravenously. In some embodiments, the anti-HER2 antibody is administered at a dose of 8 mg/kg once every 3 weeks and the anti-HER2 antibody is administered intravenously. In some embodiments, the anti-HER2 antibody is administered once every 3 weeks at a dose of 8 mg/kg for the first dose of the anti-HER2 antibody administered to the subject followed by subsequent doses of 6 mg/kg, wherein anti-HER2 antibody is administered intravenously. In some embodiments, the anti-HER2 antibody is trastuzumab and is administered at a dose of about 6 mg/kg once about every 3 weeks and the trastuzumab is administered intravenously. In some embodiments, the anti-HER2 antibody is trastuzumab and is administered at a dose of about 8 mg/kg once about every 3 weeks and the trastuzumab is administered intravenously. In some embodiments, the anti-HER2 antibody is trastuzumab and is administered once about every 3 weeks at a dose of about 8 mg/kg for the first dose of the trastuzumab administered to the subject followed by subsequent doses of about 6 mg/kg, wherein the trastuzumab is administered intravenously. In some embodiments, the anti-HER2 antibody is trastuzumab and is administered at a dose of 6 mg/kg once every 3 weeks and the trastuzumab is administered intravenously. In some embodiments, the anti-HER2 antibody is trastuzumab and is administered at a dose of 8 mg/kg once every 3 weeks and the trastuzumab is administered intravenously. In some embodiments, the anti-HER2 antibody is trastuzumab and is administered once every 3 weeks at a dose of 8 mg/kg for the first dose of trastuzumab administered to the subject followed by subsequent doses of 6 mg/kg, wherein the trastuzumab is administered intravenously. In some embodiments, the anti-HER2 antibody is trastuzumab and is administered to the subject on a 21 day treatment cycle and is administered to the subject once per treatment cycle.

D. Treatment Outcome

In some embodiments, treating the subject comprises inhibiting cancer cell growth, inhibiting cancer cell proliferation, inhibiting cancer cell migration, inhibiting cancer cell invasion, decreasing or eliminating one or more signs or symptoms of cancer, reducing the size (e.g., volume) of a cancer tumor, reducing the number of cancer tumors, reducing the number of cancer cells, inducing cancer cell necrosis, pyroptosis, oncosis, apoptosis, autophagy, or other cell death, increasing survival time of the subject, or enhancing the therapeutic effects of another drug or therapy.

In some embodiments, treating the subject as described herein results in a tumor growth inhibition (TGI) index that is between about 10% and 70% (e.g., about 10%, 15%, 20%, 25%, 30%, 35%, 40%, 45%, 50%, 55%, 60%, 65%, or 70%). Preferably, treating the subject results in a TGI index that is at least about 70% (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%). More preferably, treating the subject results in a TGI index that is at least about 85% (e.g., about 85%, 86%, 87%, 88%, 89%, 90%, 91%, 92%, 93%, 94%, 95%, 96%, 97%, 98%, 99%, or 100%). Even more preferably, treating the subject results in a TGI index that is at least about 95% (e.g., about 95%, 96%, 97%, 98%, 99%, or 100%). Most preferably, treating the subject results in a TGI index that is about 100% or more (e.g., about 100%, 101%, 102%, 103%, 104%, 105%, 106%, 107%, 108%, 109%, 110%, 111%, 112%, 113%, 114%, 115%, 116%, 117%, 118%, 119%, 120%, 125%, 130%, 135%, 140%, 145%, 150%, or more).

In particular embodiments, treating the subject with tucatinib, capecitabine, and trastuzumab results in a TGI index that is greater than the TGI index that is observed when tucatinib, capecitabine or trastuzumab is used alone. In some instances, treating the subject results in a TGI index that is greater than the TGI index that is observed when tucatinib is used alone. In other instances, treating the subject results in a TGI index that is greater than the TGI index that is observed when capecitabine is used alone. In other instances, treating the subject results in a TGI index that is greater than the TGI index that is observed when trastuzumab is used alone. In some embodiments, treating the subject results in a TGI index that is at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, or 80% greater than the TGI index that is observed when tucatinib, capecitabine or trastuzumab is used alone.

In some embodiments, the combination of the tucatinib, capecitabine and trastuzumab is synergistic. In particular embodiments, with respect to the synergistic combination, treating the subject results in a TGI index that is greater than the TGI index that would be expected if the combination of tucatinib, capecitabine and trastuzumab produced an additive effect. In some instances, the TGI index observed when a combination of tucatinib, capecitabine and trastuzumab is administered is at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, or 80% greater than the TGI index that would be expected if the combination of tucatinib, capecitabine and trastuzumab produced an additive effect.

In one aspect, a method of treating cancer with tucatinib as described herein results in an improvement in one or more therapeutic effects in the subject after administration of tucatinib relative to a baseline. In some embodiments, the one or more therapeutic effects is the size of the tumor derived from the cancer, the objective response rate, the duration of response, the time to response, progression free survival, overall survival, or any combination thereof. In one embodiment, the one or more therapeutic effects is the size of the tumor derived from the cancer. In one embodiment, the one or more therapeutic effects is decreased tumor size. In one embodiment, the one or more therapeutic effects is stable disease. In one embodiment, the one or more therapeutic effects is partial response. In one embodiment, the one or more therapeutic effects is complete response. In one embodiment, the one or more therapeutic effects is the objective response rate. In one embodiment, the one or more therapeutic effects is the duration of response. In one embodiment, the one or more therapeutic effects is the time to response. In one embodiment, the one or more therapeutic effects is progression free survival. In one embodiment, the one or more therapeutic effects is overall survival. In one embodiment, the one or more therapeutic effects is cancer regression.

In one embodiment of the methods or uses or product for uses provided herein, response to treatment with tucatinib as described herein may include the following criteria (RECIST Criteria 1.1):

| | Category | Criteria |
|---|---|---|
| Based | Complete | Disappearance of all target lesions. Any |
| on | Response | pathological lymph nodes must have reduction |
| target | (CR) | in short axis to <10 mm. |
| lesions | Partial | ≥30% decrease in the sum of the longest |
| | Response | diameter (LD) of target lesions, taking as |
| | (PR) | reference the baseline sum of LDs. |
| | Stable | Neither sufficient shrinkage to qualify for PR nor |
| | Disease | sufficient increase to qualify for PD, taking as |
| | (SD) | reference the smallest sum of LDs while in trial. |
| | Progres- | ≥20% (and ≥5 mm) increase in the sum of the LDs |
| | sive | of target lesions, taking as reference the smallest |
| | Disease | sum of the target LDs recorded while in trial or |
| | (PD) | the appearance of one or more new lesions. |
| Based | CR | Disappearance of all non-target lesions and |
| on non- | | normalization of tumor marker level. All lymph |
| target | | nodes must be non-pathological in size (<10 mm |
| lesions | | short axis). |
| | SD | Persistence of one or more non-target lesion(s) |
| | | or/and maintenance of tumor marker level above |
| | | the normal limits. |
| | PD | Appearance of one or more new lesions and/or |
| | | unequivocal progression of existing non-target |
| | | lesions. |

In one embodiment of the methods or uses or product for uses provided herein, the effectiveness of treatment with tucatinib described herein is assessed by measuring the objective response rate. In some embodiments, the objective response rate is the proportion of patients with tumor size reduction of a predefined amount and for a minimum period of time. In some embodiments the objective response rate is based upon RECIST v1.1. In one embodiment, the objective response rate is at least about 20%, at least about 25%, at least about 30%, at least about 35%, at least about 40%, at least about 45%, at least about 50%, at least about 60%, at least about 70%, or at least about 80%. In one embodiment, the objective response rate is at least about 20%-80%. In one embodiment, the objective response rate is at least about 30%-80%. In one embodiment, the objective response rate is at least about 40%-80%. In one embodiment, the objective response rate is at least about 50%-80%. In one embodiment, the objective response rate is at least about 60%-80%. In one embodiment, the objective response rate is at least about 70%-80%. In one embodiment, the objective response rate is at least about 80%. In one embodiment, the objective response rate is at least about 85%. In one embodiment, the objective response rate is at least about 90%. In one embodiment, the objective response rate is at least about 95%. In one embodiment, the objective response rate is at least about 98%. In one embodiment, the objective response rate is at least about 99%. In one embodiment, the objective response rate is at least 20%, at least 25%, at least 30%, at least 35%, at least 40%, at least 45%, at least 50%, at least 60%, at least 70%, or at least 80%. In one embodiment, the objective response rate is at least 20%-80%. In one embodiment, the objective response rate is at least 30%-80%. In one embodiment, the objective response rate is at least 40%-80%. In one embodiment, the objective response rate is at least 50%-80%. In one embodiment, the objective response rate is at least 60%-80%. In one embodiment, the objective response rate is at least 70%-80%. In one embodiment, the objective response rate is at least 80%. In one embodiment, the objective response rate is at least 85%. In one embodiment, the objective response rate is at least 90%. In one embodiment, the objective response rate is at least 95%. In one embodiment, the objective response rate is at least 98%. In one embodiment, the objective response rate is at least 99%. In one embodiment, the objective response rate is 100%.

In one embodiment of the methods or uses or product for uses provided herein, response to treatment with tucatinib described herein is assessed by measuring the size of a tumor derived from the cancer (e.g., gastric cancer, colorectal cancer, lung cancer, gall bladder cancer, or breast cancer). In one embodiment, the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of tucatinib. In one embodiment, the size of a tumor derived from the cancer is reduced by at least about 10%-80%. In one embodiment, the size of a tumor derived from the cancer is reduced by at least about 20%-80%. In one embodiment, the size of a tumor derived from the cancer is reduced by at least about 30%-80%. In one embodiment, the size of a tumor derived from the cancer is reduced by at least about 40%-80%. In one embodiment, the size of a tumor derived from the cancer is reduced by at least about 50%-80%. In one embodiment, the size of a tumor derived from the cancer is reduced by at least about 60%-80%. In one embodiment, the size of a tumor derived from the cancer is reduced by at least about 70%-80%. In one embodiment, the size of a tumor derived from the cancer is reduced by at least about 80%. In one embodiment, the size of a tumor derived from the cancer is reduced by at least about 85%. In one embodiment, the size of a tumor derived from the cancer is reduced by at least about 90%. In one embodiment, the size of a tumor derived from the cancer is reduced by at least about 95%. In one embodiment, the size of a tumor derived from the cancer is reduced by at least about 98%. In one embodiment, the size of a tumor derived from the cancer is reduced by at least about 99%. In one embodiment, the size of a tumor derived from the cancer is reduced by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of tucatinib. In one embodiment, the size of a tumor derived from the cancer is reduced by at least 10%-80%. In one embodiment, the size of a tumor derived from the cancer is reduced by at least 20%-80%. In one embodiment, the size of a tumor derived from the cancer is reduced by at least 30%-80%. In one embodiment, the size of a tumor derived from the cancer is reduced by at least 40%-80%. In one embodiment, the size of a tumor derived from the cancer is reduced by at least 50%-80%. In one embodiment, the size of a tumor derived from the cancer is reduced by at least 60%-80%. In one embodiment, the size of a tumor derived from the cancer is reduced by at least 70%-80%. In one embodiment, the size of a tumor derived from the cancer is reduced by at least 80%. In one embodiment, the size of a tumor derived from the cancer is reduced by at least 85%. In one embodiment, the size of a tumor derived from the cancer is reduced by at least 90%. In one embodiment, the size of a tumor derived from the cancer is reduced by at least 95%. In one embodiment, the size of a tumor derived from the cancer is reduced by at least 98%. In one embodiment, the size of a tumor derived from the cancer is reduced by at least 99%. In one embodiment, the size of a tumor derived from the cancer is reduced by 100%. In one embodiment, the size of a tumor derived from the cancer is measured by magnetic resonance imaging (MRI). In one embodiment, the size of a tumor derived from the cancer is measured by computed tomography (CT). In one embodiment, the size of a tumor derived from the cancer is measured by positron emission tomography (PET). In one embodiment, the size of a tumor derived from the cancer is measured by mammography. In one embodiment, the size of a tumor derived from the cancer is measured by sonography. See Gruber et. al., 2013, BMC Cancer. 13:328.

In one embodiment of the methods or uses or product for uses provided described herein, response to treatment with tucatinib described herein, promotes regression of a tumor derived from the cancer (e.g., gastric cancer, colorectal cancer, lung cancer, gall bladder cancer, or breast cancer). In one embodiment, a tumor derived from the cancer regresses by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the tucatinib described herein. In one embodiment, a tumor derived from the cancer regresses by at least about 10% to about 80%. In one embodiment, a tumor derived from the cancer regresses by at least about 20% to about 80%. In one embodiment, a tumor derived from the cancer regresses by at least about 30% to about 80%. In one embodiment, a tumor derived from the cancer regresses by at least about 40% to about 80%. In one embodiment, a tumor derived from the cancer regresses by at least about 50% to about 80%. In one embodiment, a tumor derived from the cancer regresses by at least about 60% to about 80%. In one embodiment, a tumor derived from the cancer regresses by at least about 70% to about 80%. In one embodiment, a tumor derived from the cancer regresses by at least about 80%. In one embodiment, a tumor derived from the cancer regresses by at least about 85%. In one embodiment, a tumor derived from the cancer regresses by at least about 90%. In one embodiment, a tumor derived from the cancer regresses by at least about 95%. In one embodiment, a tumor derived from the cancer regresses by at least about 98%. In one embodiment, a tumor derived from the cancer regresses by at least about 99%. In one embodiment, a tumor derived from the cancer regresses by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of tucatinib described herein. In one embodiment, a tumor derived from the cancer regresses by at least 10% to 80%. In one embodiment, a tumor derived from the cancer regresses by at least 20% to 80%. In one embodiment, a tumor derived from the cancer regresses by at least 30% to 80%. In one embodiment, a tumor derived from the cancer regresses by at least 40% to 80%. In one embodiment, a tumor derived from the cancer regresses by at least 50% to 80%. In one embodiment, a tumor derived from the cancer regresses by at least 60% to 80%. In one embodiment, a tumor derived from the cancer regresses by at least 70% to 80%. In one embodiment, a tumor derived from the cancer regresses by at least 80%. In one embodiment, a tumor derived from the cancer regresses by at least 85%. In one embodiment, a tumor derived from the cancer regresses by at least 90%. In one embodiment, a tumor derived from the cancer regresses by at least 95%. In one embodiment, a tumor derived from the cancer regresses by at least 98%. In one embodiment, a tumor derived from the cancer regresses by at least 99%. In one embodiment, a tumor derived from the cancer regresses by 100%. In one embodiment, regression of a tumor is determined by magnetic resonance imaging (MRI). In one embodiment, regression of a tumor is determined by computed tomography (CT). In one embodiment, regression of a tumor is determined by positron emission tomography (PET). In one embodiment, regression of a tumor is determined by mammography. In one embodiment, regression of a tumor is determined by sonography. See Gruber et. al., 2013, BMC Cancer. 13:328.

In one embodiment of the methods or uses or product for uses described herein, response to treatment with tucatinib described herein is assessed by measuring the time of progression free survival after administration of tucatinib. In some embodiments, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of tucatinib. In some embodiments, the subject exhibits progression-free survival of at least about 6 months after administration of tucatinib. In some embodiments, the subject exhibits progression-free survival of at least about one year after administration of tucatinib. In some embodiments, the subject exhibits progression-free survival of at least about two years after administration of tucatinib. In some embodiments, the subject exhibits progression-free survival of at least about three years after administration of tucatinib. In some embodiments, the subject exhibits progression-free survival of at least about four years after administration of tucatinib. In some embodiments, the subject exhibits progression-free survival of at least about five years after administration of tucatinib. In some embodiments, the subject exhibits progression-free survival of at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of tucatinib. In some embodiments, the subject exhibits progression-free survival of at least 6 months after administration of tucatinib. In some embodiments, the subject exhibits progression-free survival of at least one year after administration of tucatinib. In some embodiments, the subject exhibits progression-free survival of at least two years after administration of tucatinib. In some embodiments, the subject exhibits progression-free survival of at least three years after administration of tucatinib. In some embodiments, the subject exhibits progression-free survival of at least four years after administration of tucatinib. In some embodiments, the subject exhibits progression-free survival of at least five years after administration of tucatinib.

In one embodiment of the methods or uses or product for uses described herein, response to treatment with tucatinib described herein is assessed by measuring the time of overall survival after administration of tucatinib. In some embodiments, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of tucatinib. In some embodiments, the subject exhibits overall survival of at least about 6 months after administration of tucatinib. In some embodiments, the subject exhibits overall survival of at least about one year after administration of tucatinib. In some embodiments, the subject exhibits overall survival of at least about two years after administration of tucatinib. In some embodiments, the subject exhibits overall survival of at least about three years after administration of tucatinib. In some embodiments, the subject exhibits overall survival of at least about four years after administration of tucatinib. In some embodiments, the subject exhibits overall survival of at least about five years after administration of tucatinib. In some embodiments, the subject exhibits overall survival of at least 1 month, at least 2 months, at least 3 months, at least 4 months, at least 5 months, at least 6 months, at least 7 months, at least 8 months, at least 9 months, at least 10 months, at least 11 months, at least about 12 months, at least eighteen months, at least two years, at least three years, at least four years, or at least five years after administration of tucatinib. In some embodiments, the subject exhibits overall survival of at least 6 months after administration of tucatinib. In some embodiments, the subject exhibits overall survival of at least one year after administration of tucatinib. In some embodiments, the subject exhibits overall survival of at least two years after administration of tucatinib. In some embodiments, the subject exhibits overall survival of at least three years after administration of tucatinib. In some embodiments, the subject exhibits overall survival of at least four years after administration of tucatinib. In some embodiments, the subject exhibits overall survival of at least five years after administration of tucatinib.

In one embodiment of the methods or uses or product for uses described herein, response to treatment with tucatinib described herein is assessed by measuring the duration of response to tucatinib after administration of tucatinib. In some embodiments, the duration of response to tucatinib is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of tucatinib. In some embodiments, the duration of response to tucatinib is at least about 6 months after administration of tucatinib. In some embodiments, the duration of response to tucatinib is at least about one year after administration of tucatinib. In some embodiments, the duration of response to tucatinib is at least about two years after administration of tucatinib. In some embodiments, the duration of response to tucatinib is at least about three years after administration of tucatinib. In some embodiments, the duration of response to tucatinib is at least about four years after administration of tucatinib. In some embodiments, the duration of response to tucatinib is at least about five years after administration of tucatinib. In some embodiments, the duration of response to tucatinib is at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of tucatinib. In some embodiments, the duration of response to tucatinib is at least 6 months after administration of tucatinib. In some embodiments, the duration of response to tucatinib is at least one year after administration of tucatinib. In some embodiments, the duration of response to tucatinib is at least two years after administration of tucatinib. In some embodiments, the duration of response to tucatinib is at least three years after administration of tucatinib. In some embodiments, the duration of response to tucatinib is at least four years after administration of tucatinib. In some embodiments, the duration of response to tucatinib is at least five years after administration of tucatinib.

E. Compositions

In another aspect, the present invention provides a pharmaceutical composition comprising tucatinib and a pharmaceutically acceptable carrier. In another aspect, the present invention provides a pharmaceutical composition comprising capecitabine and a pharmaceutically acceptable carrier. In another aspect, the present invention provides a pharmaceutical composition comprising an anti-HER2 antibody and a pharmaceutically acceptable carrier. In another aspect, the present invention provides a pharmaceutical composition comprising tucatinib, capecitabine, and a pharmaceutically acceptable carrier. In another aspect, the present invention provides a pharmaceutical composition comprising tucatinib, an anti-HER2 antibody, and a pharmaceutically acceptable carrier. In another aspect, the present invention provides a pharmaceutical composition comprising capecitabine, an anti-HER2 antibody, and a pharmaceutically acceptable carrier. In another aspect, the present invention provides a pharmaceutical composition comprising tucatinib, capecitabine, an anti-HER2 antibody, and a pharmaceutically acceptable carrier. In some embodiments, the anti-HER2 antibody is a member selected from the group consisting of trastuzumab, pertuzumab, ado-trastuzumab emtansine, margetuximab, and a combination thereof. In some instances, the anti-HER2 antibody is a combination of trastuzumab and pertuzumab. In some embodiments, the anti-HER2 antibody is trastuzumab.

In some embodiments, tucatinib is present at a concentration between about 0.1 nM and 10 nM (e.g., about 0.1, 0.2, 0.3, 0.4, 0.5 0.6, 0.7, 0.8, 0.9, 1.0, 1.5, 2, 2.5, 3, 3.5, 4, 4.5, 5, 5.5, 6, 6.5, 7, 7.5, 8, 8.5, 9, 9.5, or 10 nM). In other embodiments, tucatinib is present at a concentration between about 10 nM and 100 nM (e.g., about 10, 15, 20, 25, 30, 35, 40, 45, 50, 55, 60, 65, 70, 75, 80, 85, 90, 95, or 100 nM). In some other embodiments, tucatinib is present at a concentration between about 100 nM and 1,000 nM (e.g., about 100, 150, 200, 250, 300, 350, 400, 450, 500, 550, 600, 650, 700, 750, 800, 850, 900, 950, or 1,000 nM). In yet other embodiments, tucatinib is present at a concentration at least about 1,000 nM to 10,000 nM (e.g., at least about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000, or more nM).

In some embodiments, the anti-HER2 antibody is present at a concentration between about 0.1 nM and 10 nM (e.g., about 0.1, 0.2, 0.3, 0.4, 0.5 0.6, 0.7, 0.8, 0.9, 1.0, 1.5, 2, 2.5, 3, 3.5, 4, 4.5, 5, 5.5, 6, 6.5, 7, 7.5, 8, 8.5, 9, 9.5, or 10 nM). In other embodiments, the anti-HER2 antibody is present at a concentration between about 10 nM and 100 nM (e.g., about 10, 15, 20, 25, 30, 35, 40, 45, 50, 55, 60, 65, 70, 75, 80, 85, 90, 95, or 100 nM). In some other embodiments, the anti-HER2 antibody is present at a concentration between about 100 nM and 1,000 nM (e.g., about 100, 150, 200, 250, 300, 350, 400, 450, 500, 550, 600, 650, 700, 750, 800, 850, 900, 950, or 1,000 nM). In yet other embodiments, the anti-HER2 antibody is present at a concentration of at least about 1,000 nM to 10,000 nM (e.g., at least about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000, or more nM).

In some embodiments, capecitabine is present at a concentration between about 0.1 nM and 10 nM (e.g., about 0.1, 0.2, 0.3, 0.4, 0.5 0.6, 0.7, 0.8, 0.9, 1.0, 1.5, 2, 2.5, 3, 3.5, 4, 4.5, 5, 5.5, 6, 6.5, 7, 7.5, 8, 8.5, 9, 9.5, or 10 nM). In other embodiments, capecitabine is present at a concentration between about 10 nM and 100 nM (e.g., about 10, 15, 20, 25, 30, 35, 40, 45, 50, 55, 60, 65, 70, 75, 80, 85, 90, 95, or 100 nM). In some other embodiments, capecitabine is present at a concentration between about 100 nM and 1,000 nM (e.g., about 100, 150, 200, 250, 300, 350, 400, 450, 500, 550, 600, 650, 700, 750, 800, 850, 900, 950, or 1,000 nM). In yet other embodiments, capecitabine is present at a concentration of at least about 1,000 nM to 10,000 nM (e.g., at least about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000, or more nM).

The pharmaceutical compositions of the present invention may be prepared by any of the methods well-known in the art of pharmacy. Pharmaceutically acceptable carriers suitable for use with the present invention include any of the standard pharmaceutical carriers, buffers and excipients, including phosphate-buffered saline solution, water, and emulsions (such as an oil/water or water/oil emulsion), and various types of wetting agents or adjuvants. Suitable pharmaceutical carriers and their formulations are described in Remington's Pharmaceutical Sciences (Mack Publishing Co., Easton, 19th ed. 1995). Preferred pharmaceutical carriers depend upon the intended mode of administration of the active agent.

The pharmaceutical compositions of the present invention can include a combination of drugs (e.g., tucatinib, capecitabine, and an anti-HER2 antibody), or any pharmaceutically acceptable salts thereof, as active ingredients and a pharmaceutically acceptable carrier or excipient or diluent. A pharmaceutical composition may optionally contain other therapeutic ingredients.

The compositions (e.g., comprising tucatinib, capecitabine, an anti-HER2 antibody, or a combination thereof) can be combined as the active ingredients in intimate admixture with a suitable pharmaceutical carrier or excipient according to conventional pharmaceutical compounding techniques. Any carrier or excipient suitable for the form of preparation desired for administration is contemplated for use with the compounds disclosed herein.

The pharmaceutical compositions include those suitable for oral, topical, parenteral, pulmonary, nasal, or rectal administration. The most suitable route of administration in any given case will depend in part on the nature and severity of the cancer condition and also optionally the HER2 status or stage of the cancer.

Other pharmaceutical compositions include those suitable for systemic (e.g., enteral or parenteral) administration. Systemic administration includes oral, rectal, sublingual, or sublabial administration. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In particular embodiments, pharmaceutical compositions of the present invention may be administered intratumorally.

Compositions for pulmonary administration include, but are not limited to, dry powder compositions consisting of the powder of a compound described herein (e.g., tucatinib, capecitabine, an anti-HER2 antibody, or a combination thereof), or a salt thereof, and the powder of a suitable carrier or lubricant. The compositions for pulmonary administration can be inhaled from any suitable dry powder inhaler device known to a person skilled in the art.

Compositions for systemic administration include, but are not limited to, dry powder compositions consisting of the composition as set forth herein (e.g., tucatinib, capecitabine, an anti-HER2 antibody, or a combination thereof) and the powder of a suitable carrier or excipient. The compositions for systemic administration can be represented by, but not limited to, tablets, capsules, pills, syrups, solutions, and suspensions.

In some embodiments, the compositions (e.g., tucatinib, capecitabine, an anti-HER2 antibody, or a combination thereof) further include a pharmaceutical surfactant. In other embodiments, the compositions further include a cryoprotectant. In some embodiments, the cryoprotectant is selected from the group consisting of glucose, sucrose, trehalose, lactose, sodium glutamate, PVP, HPβCD, CD, glycerol, maltose, mannitol, and saccharose.

Pharmaceutical compositions or medicaments for use in the present invention can be formulated by standard techniques using one or more physiologically acceptable carriers or excipients. Suitable pharmaceutical carriers are described herein and in Remington: The Science and Practice of Pharmacy, 21st Ed., University of the Sciences in Philadelphia, Lippencott Williams & Wilkins (2005).

Controlled-release parenteral formulations of the compositions (e.g., tucatinib, capecitabine, an anti-HER2 antibody, or a combination thereof) can be made as implants, oily injections, or as particulate systems. For a broad overview of delivery systems see Banga, A. J., Therapeutic Peptides and Proteins: Formulation, Processing, and Delivery Systems, Technomic Publishing Company, Inc., Lancaster, Pa., (1995), which is incorporated herein by reference. Particulate systems include microspheres, microparticles, microcapsules, nanocapsules, nanospheres, and nanoparticles.

Polymers can be used for ion-controlled release of compositions of the present invention. Various degradable and nondegradable polymeric matrices for use in controlled drug delivery are known in the art (Langer R., Accounts Chem. Res., 26:537-542 (1993)). For example, the block copolymer, polaxamer 407 exists as a viscous yet mobile liquid at low temperatures but forms a semisolid gel at body temperature. It has been shown to be an effective vehicle for formulation and sustained delivery of recombinant interleukin 2 and urease (Johnston et al., Pharm. Res., 9:425-434 (1992); and Pec et al., J. Parent. Sci. Tech., 44(2):58 65 (1990)). Alternatively, hydroxyapatite has been used as a microcarrier for controlled release of proteins (Ijntema et al., Int. J. Pharm., 112:215-224 (1994)). In yet another aspect, liposomes are used for controlled release as well as drug targeting of the lipid-capsulated drug (Betageri et al., LIPOSOME DRUG DELIVERY SYSTEMS, Technomic Publishing Co., Inc., Lancaster, Pa. (1993)). Numerous additional systems for controlled delivery of therapeutic proteins are known. See, e.g., U.S. Pat. Nos. 5,055,303, 5,188,837, 4,235,871, 4,501,728, 4,837,028 4,957,735 and 5,019,369, 5,055,303; 5,514,670; 5,413,797; 5,268,164; 5,004,697; 4,902,505; 5,506,206, 5,271,961; 5,254,342 and 5,534,496, each of which is incorporated herein by reference.

For oral administration of a combination of tucatinib, capecitabine, and/or an anti-HER2 antibody, a pharmaceutical composition or a medicament can take the form of, for example, a tablet or a capsule prepared by conventional means with a pharmaceutically acceptable excipient. The present invention provides tablets and gelatin capsules comprising tucatinib, capecitabine, an anti-HER2 antibody, or a combination thereof, or a dried solid powder of these drugs, together with (a) diluents or fillers, e.g., lactose, dextrose, sucrose, mannitol, sorbitol, cellulose (e.g., ethyl cellulose, microcrystalline cellulose), glycine, pectin, polyacrylates or calcium hydrogen phosphate, calcium sulfate, (b) lubricants, e.g., silica, talcum, stearic acid, magnesium or calcium salt, metallic stearates, colloidal silicon dioxide, hydrogenated vegetable oil, corn starch, sodium benzoate, sodium acetate or polyethyleneglycol; for tablets also (c) binders, e.g., magnesium aluminum silicate, starch paste, gelatin, tragacanth, methylcellulose, sodium carboxymethylcellulose, polyvinylpyrrolidone or hydroxypropyl methylcellulose; if desired (d) disintegrants, e.g., starches (e.g., potato starch or sodium starch), glycolate, agar, alginic acid or its sodium salt, or effervescent mixtures; (e) wetting agents, e.g., sodium lauryl sulphate, or (f) absorbents, colorants, flavors and sweeteners.

Tablets may be either film coated or enteric coated according to methods known in the art. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, or sweetening agents as appropriate. If desired, preparations for oral administration can be suitably formulated to give controlled release of the active compound(s).

Typical formulations for topical administration of tucatinib, capecitabine, an anti-HER2 antibody, or a combination thereof include creams, ointments, sprays, lotions, and patches. The pharmaceutical composition can, however, be formulated for any type of administration, e.g., intradermal, subdermal, intravenous, intramuscular, subcutaneous, intranasal, intracerebral, intratracheal, intraarterial, intraperitoneal, intravesical, intrapleural, intracoronary or intratumoral injection, with a syringe or other devices. Formulation for administration by inhalation (e.g., aerosol), or for oral or rectal administration is also contemplated.

Suitable formulations for transdermal application include an effective amount of one or more compounds described herein, optionally with a carrier. Preferred carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used.

The compositions and formulations set forth herein (e.g., tucatinib, capecitabine, an anti-HER2 antibody, or a combination thereof) can be formulated for parenteral administration by injection, for example by bolus injection or continuous infusion. Formulations for injection can be presented in unit dosage form, for example, in ampules or in multi-dose containers, with an added preservative. Injectable compositions are preferably aqueous isotonic solutions or suspensions, and suppositories are preferably prepared from fatty emulsions or suspensions. The compositions may be sterilized or contain adjuvants, such as preserving, stabilizing, wetting or emulsifying agents, solution promoters, salts for regulating the osmotic pressure or buffers. Alternatively, the active ingredient(s) can be in powder form for constitution with a suitable vehicle, for example, sterile pyrogen-free water, before use. In addition, they may also contain other therapeutically valuable substances. The compositions are prepared according to conventional mixing, granulating or coating methods, respectively.

For administration by inhalation, the compositions (e.g., comprising tucatinib, capecitabine, an anti-HER2 antibody, or a combination thereof) may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound(s) and a suitable powder base, for example, lactose or starch.

The compositions (e.g., comprising tucatinib, capecitabine, an anti-HER2 antibody, or a combination thereof) can also be formulated in rectal compositions, for example, suppositories or retention enemas, for example, containing conventional suppository bases, for example, cocoa butter or other glycerides.

Furthermore, the active ingredient(s) can be formulated as a depot preparation. Such long-acting formulations can be administered by implantation (for example, subcutaneously or intramuscularly) or by intramuscular injection. Thus, for example, one or more of the compounds described herein can be formulated with suitable polymeric or hydrophobic materials (for example as an emulsion in an acceptable oil) or ion exchange resins, or as sparingly soluble derivatives, for example, as a sparingly soluble salt.

F. Articles of Manufacture and Kits

In another aspect, the present invention provides an article of manufacture or kit for treating or ameliorating the effects of breast cancer in a subject, the article of manufacture or kit comprising a pharmaceutical composition of the present invention (e.g., a pharmaceutical composition comprising tucatinib, capecitabine, an anti-HER2 antibody, or a combination thereof). In some embodiments, the anti-HER2 antibody is trastuzumab, pertuzumab, ado-trastuzumab emtansine, margetuximab, or a combination thereof. In some instances, the anti-HER2 antibody is a combination of trastuzumab and pertuzumab. In some embodiments, the anti-HER2 antibody is trastuzumab.

The articles of manufacture or kits are suitable for treating or ameliorating the effects of cancers, particularly cancers that have been determined to express a mutant form of HER2. In some embodiments, the cancer is an advanced cancer.

Materials and reagents to carry out the various methods of the present invention can be provided in articles of manufacture or kits to facilitate execution of the methods. As used herein, the term “kit” includes a combination of articles that facilitates a process, assay, analysis, or manipulation. In particular, kits of the present invention find utility in a wide range of applications including, for example, diagnostics, prognostics, therapy, and the like.

Articles of manufacture or kits can contain chemical reagents as well as other components. In addition, the articles of manufacture or kits of the present invention can include, without limitation, instructions to the user, apparatus and reagents for administering combinations of tucatinib, capecitabine and anti-HER2 antibodies or pharmaceutical compositions thereof, sample tubes, holders, trays, racks, dishes, plates, solutions, buffers, or other chemical reagents. In some embodiments, the articles of manufacture or kits contain instructions, apparatus, or reagents for determining the genotype of a gene (e.g., KRAS, NRAS, BRAF) or determining the expression of HER2 in a sample. Articles of manufacture or kits of the present invention can also be packaged for convenient storage and safe shipping, for example, in a box having a lid.

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1: In Vivo Efficacy Study of Tucatinib in the Treatment of HuPrime® PDX GL1208 Gallbladder Cancer Xenograft Model in Female BALB/c Nude Mice

The objective of this study was to evaluate preclinically the in vivo therapeutic efficacy of tucatinib in the treatment of the Huprime® PDX GL1208 gallbladder cancer xenograft model in female BALB/c nude mice. The Huprime® PDX GL1208 gallbladder cancer xenograft model has an S310Y HER2 mutation.

Tumor fragments from stock mice were harvested and used for inoculation into mice. Each mouse was inoculated subcutaneously in the right front flank with primary human tumor xenograft model GL1208 tumor fragment (2-3 mm in diameter) for tumor development.

32 mice were enrolled in the study. All animals were randomly allocated to 4 study groups as follows:

| | | | Dose | | Dosing | Dosing |
|---|---|---|---|---|---|---|
| | | | level | Dose | volume | frequency & |
| Group | No. | Treatment | (mg/kg) | route | (μL/G) | duration |
| 1 | 8 | Vehicle | — | p.o | 10 | BID X 28 |
| 2 | 8 | Tucatinib | 50 | p.o | 10 | BID X 28 |
| 3 | 8 | Trastuzumab | 20 | i.p. | 10 | QW X 4 |
| 4 | 8 | Trastuzumab | 20 | i.p. | 10 | QW X 4 |
| | | Tucatinib | 50 | p.o | 10 | BID X 28 |

p.o. = oral, i.p. = intraperitoneal, BID X 28 = twice a day for 28 days, and QW X 4 = once a week for 4 weeks.

The randomization was started when the mean tumor size reached approximately 146 mm³. Randomization was performed based on “Matched distribution” method (Study Director™ software, version 3.1.399.19). The date of randomization was denoted as day 0.

After tumor tissues' inoculation, the animals were checked daily for morbidity and mortality. During routine monitoring, the animals were checked for any effects of tumor growth and treatments on behavior such as mobility, food and water consumption, body weight gain/loss (Body weights would be measured twice per week after randomization), eye/hair matting and any other abnormalities. Mortality and observed clinical signs were recorded for individual animals in detail.

Tumor volumes were measured twice per week after randomization in two dimensions using a caliper, and the volume was expressed in mm3 using the formula: V=(L×W×W)/2, where V is tumor volume, L is tumor length (the longest tumor dimension) and W is tumor width (the longest tumor dimension perpendicular to L). Dosing as well as tumor and body weight measurements were conducted in a Laminar Flow Cabinet.

The body weights and tumor volumes were measured by using Study Director™ software (version 3.1.399.19).

The treatment was initiated one day post grouping (day 1).

Drugs were formulated as described in the following table:

| | Conc. | Dose | | |
|---|---|---|---|---|
| Drug | (mg/mL) | (mg/kg) | Formulation Details and preparation frequency | Storage |
| Vehicle | 0 | 0 | Control dosing solution was 30% Captisol in | Stored |
| | | | deionized water with the pH adjusted to 3-3.5 using | at −20° C. |
| | | | 5N HCl. | |
| Trastuzumab | 2 mg/ml | 20 mg/kg | Trastuzumab was supplied as a lyophilized powder. | Stored |
| 20 mg/kg | | | Material was reconstituted with sterile water for | at −20° C. |
| | | | injection as described in the package insert using 7.4 | |
| | | | ml of sterile water for injection to yield a 21 mg/ml | |
| | | | solution. This stock solution was stored in single use | |
| | | | aliquots at −80° C. Single use aliquots that were | |
| | | | thawed on the day of dosing, then diluted using sterile | |
| | | | saline to 2 mg/ml. Concentration was 2.0 mg/ml. | |
| Tucatinib | 5 mg/ml | 50 mg/kg | Prepared dosing solutions weekly and made daily use | Stored |
| 50 mg/kg | | | aliquots. Stored daily aliquots at −20° C. Protected | at −20° C. |
| | | | both powder and formulated material from light. | |
| | | | A dosing solution with a concentration of 5 mg/ml | |
| | | | was prepared once weekly according to the following | |
| | | | protocol: | |
| | | | 1) Weighed out ONT-380 (tucatinib) powder enough | |
| | | | for seven days of dosing. | |
| | | | 2) Added 90% of total volume of 30% Captisol | |
| | | | solution. | |
| | | | 3) Stirred for a minimum of 45 minutes to form | |
| | | | uniform suspension (without heating). Vortexed every | |
| | | | 15 minutes to remove any particulates from the vessel | |
| | | | wall. | |
| | | | 4) Acidified to approximately pH 3.5 using 5N HCl | |
| | | | and documented the amount of HCl used. Reduced | |
| | | | the final volume of vehicle to be added later by the | |
| | | | amount of HCl used. | |
| | | | 5) Stirred for 2 hours (without heating) or until the | |
| | | | solution became clear. Checked occasionally for | |
| | | | particulates on vessel wall and vortexed to remove | |
| | | | any particulates (inverted vessel if content hit the | |
| | | | cap). | |
| | | | 6) Checked pH and adjusted pH with 5N HCl to | |
| | | | approximately 3.5 if necessary. | |
| | | | 7) Added remainder of 30% Captisol to obtain the | |
| | | | final volume. | |
| | | | 8) Stirred and vortexed occasionally if needed for | |
| | | | another 30 minutes until the solution was clear and no | |
| | | | particulates are visible on the walls of the vessel | |
| | | | (invert vessel if content hit the cap). | |
| | | | 9) Freeze each time aliquots (enough for morning or | |
| | | | afternoon dosing) at −20° C. | |
| | | | 10) Thawed aliquot at room temperature for 15 | |
| | | | minutes or until completely thawed and reached room | |
| | | | temperature. Vortex for 30 seconds and checked if | |
| | | | aliquot was a clear solution. If cloudy, vortex until | |
| | | | clear solution was obtained. During application of | |
| | | | therapy, kept the dosing solution for the other animals | |
| | | | on a stirrer. | |

### Study Endpoints

Tumor growth inhibition (TGI): TGI % was an indication of antitumor activity, and expressed as: TGI (%)=100×(1−T/C). T and C were the mean tumor volume of the treated and control groups, respectively, on a given day.

Statistical analysis of the difference in mean tumor volume among the groups was conducted using the methods below. The data collected on the last dosing/observation day for every single group was used despite diverse individual termination dates.

### Statistical Analysis

To compare tumor volumes of different groups at a pre-specified day, Bartlett's test was used to check the assumption of homogeneity of variance across all groups. When the p-value of Bartlett's test was >=0.05, one-way ANOVA was run to test overall equality of means across all groups. If the p-value of the one-way ANOVA was <0.05, post hoc testing was performed by running Tukey's HSD (honest significant difference) tests for all pairwise comparisons, and Dunnett's tests for comparing each treatment group with the vehicle group. When the p-value of Bartlett's test was <0.05, the Kruskal-Wallis test was run to test overall equality of medians among all groups. If the p-value the Kruskal-Wallis test was <0.05, post hoc testing was performed by running Conover's non-parametric test for all pairwise comparisons or for comparing each treatment group with the vehicle group, both with single-step p-value adjustment.

All statistical analyses were done in R-a language and environment for statistical computing and graphics (version 3.3.1). All tests were two-sided unless otherwise specified, and p-values of <0.05 were regarded as statistically significant.

### Results

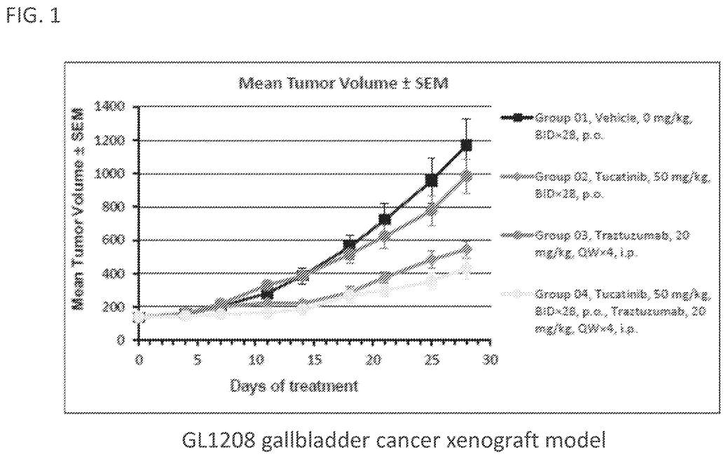

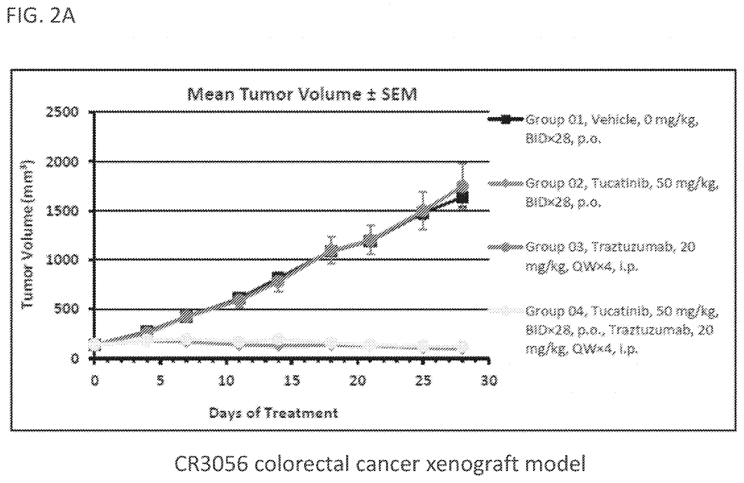

The mean tumor volume over time (±SEM) for each treatment group is shown in FIG. 1. Tumor growth inhibition (TGI) with data collected on day 28:

| | | Tumor size | TGI | T/C |
|---|---|---|---|---|
| Group | Treatment description | (mm³)^{a} on day 28 | (%)^{b} | (%)^{b} |
| 1 | Vehicle | 1167.41 ± 157.90 | — | — |
| 2 | Tucatinib (50 mg/kg) | 543.98 ± 53.25 | 53.40 | 46.60 |
| 3 | Traztuzumab (20 mg/kg) | 985.29 ± 100.49 | 15.60 | 84.40 |
| 4 | Tucatinib (50 mg/kg) + | 430.00 ± 67.17 | 63.17 | 36.83 |
| | Traztuzumab (20 mg/kg) | | | |

Note:
^{a}Mean ± SEM;
^{b}Used mean Tumor Volume for TGI, and T/C calculation: T/C = mean (T28)/mean (C28) * 100%; TGI = (mean (C28) − mean (T28))/mean (C28) * 100%.

Statistical analysis of tumor volume with data collected on day 28:

| Test | P values | Significance level |
|---|---|---|
| Bartlett's test | 0.0293 | * |
| Kruskal-Wallis test | 0.000155 | *** |
| G01-G02 | 0.000331 | *** |
| G01-G03 | 0.946 | ns |
| G01-G04 | 8.95e−06 | *** |
| G02-G03 | 0.00133 | ** |
| G02-G04 | 0.657 | ns |
| G03-G04 | 3.78e−05 | *** |

Note:
ns = no significance

### Result Summary

In this study, the therapeutic efficacy of tucatinib as a single agent or in combination with trastuzumab in the treatment of subcutaneous gallbladder cancer PDX xenograft model GL1208 in female BALB/c nude mice was evaluated.

On day 28, the test compound tucatinib at 50 mg/kg (Group 2) as a single agent or tucatinib at 50 mg/kg in combination with traztuzumab at 20 mg/kg (Group 4) both produced a statistical significant anti-tumor efficacy (TGI=53.40%, and 63.17%, respectively; both P <0.001) compared to vehicle treatment group (group 1).

However, on day 28, traztuzumab 20 mg/kg (Group 3) as a single agent didn't produce a statistical significant anti-tumor efficacy (TGI=15.60%, and P >0.05) compared to vehicle treatment group (group 1).

In summary, tucatinib at 50 mg/kg as a single agent or tucatinib at 50 mg/kg in combination with traztuzumab at 20 mg/kg both showed significant antitumor efficacy in subcutaneous gallbladder cancer PDX xenograft model GL1208 in female BALB/c nude mice in this study.

### Example 2: In Vivo Efficacy Study of Tucatinib in the Treatment of HuPrime® PDX CR3056 Colorectal Cancer Xenograft Model in Female BALB/c Nude Mice

The objective of this study was to evaluate preclinically the in vivo therapeutic efficacy of tucatinib in the treatment of the Huprime® PDX CR3056 colorectal cancer xenograft model in female BALB/c nude mice. The Huprime® PDX CR3056 colorectal cancer xenograft model has both HER2 amplification and a V777L mutation in HER2.

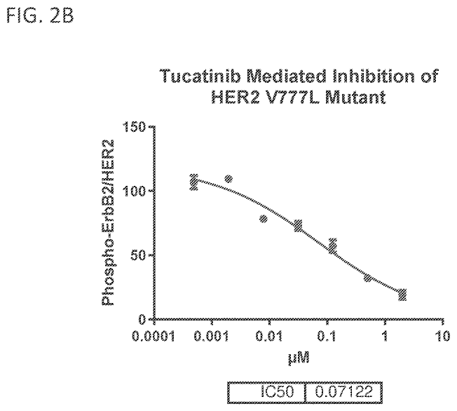

Experiments and analysis were conducted as described in Example 1, with the exception of the Huprime® PDX CR3056 colorectal cancer xenograft model being used instead of the Huprime® PDX GL1208 gallbladder cancer xenograft model. In addition, the ability of tucatinib to inhibit the kinase activity of HER2 V777L was assessed in vitro. As shown in FIG. 2B, tucatinib potently inhibits the kinase activity of the V777L activating mutation in vitro with an IC50 of 0.07122 μM.

### Results

The mean tumor volume over time (±SEM) for each treatment group is shown in FIG. 2A. Tumor growth inhibition (TGI) with data collected on day 28:

| | | Tumor size | TGI | T/C |
|---|---|---|---|---|
| Group | Treatment description | (mm³)^{a} on day 28 | (%)^{b} | (%)^{b} |
| 1 | Vehicle | 1636.76 ± 89.89 | — | — |
| 2 | Tucatinib (50 mg/kg) | 94.93 ± 8.79 | 94.20 | 5.80 |
| 3 | Traztuzumab (20 mg/kg) | 1747.60 ± 227.60 | −6.77 | 106.77 |
| 4 | Tucatinib (50 mg/kg) + | 118.69 ± 32.79 | 92.75 | 7.25 |
| | Traztuzumab (20 mg/kg) | | | |

Note:
^{a}Mean ± SEM;
^{b}Used mean Tumor Volume for TGI, and T/C calculation: T/C = mean (T28)/mean (C28) * 100%; TGI = (mean (C28) − mean (T28))/mean (C28) * 100%.

Statistical analysis of tumor volume with data collected on day 28:

| Test | P values | Significance level |
|---|---|---|
| Bartlett's test | 1.61e−10 | *** |
| Kruskal-Wallis test | 3.27e−05 | *** |
| G01-G02 | 9.15e−06 | *** |
| G01-G03 | 0.889 | ns |
| G01-G04 | 4.04e−06 | *** |
| G02-G03 | 1.37e−06 | *** |
| G02-G04 | 0.99 | ns |
| G03-G04 | 6.19e−07 | *** |

Note:
ns = no significance

### Result Summary

In this study, the therapeutic efficacy of tucatinib as a single agent or in combination with trastuzumab in the treatment of subcutaneous colorectal cancer PDX xenograft model CR3056 in female BALB/c nude mice was evaluated.

On day 28, the test compound tucatinib at 50 mg/kg (Group 2) as a single agent or tucatinib at 50 mg/kg in combination with traztuzumab at 20 mg/kg (Group 4) both produced a statistical significant anti-tumor efficacy (TGI=94.20%, and 92.75%, respectively; both P <0.001) compared to vehicle treatment group (Group 1).

However, on day 28, traztuzumab 20 mg/kg (Group 3) as a single agent didn't produce a statistical significant anti-tumor efficacy (TGI=−6.77%, and P >0.05) compared to vehicle treatment group (Group 1).

In summary, tucatinib at 50 mg/kg as a single agent or tucatinib at 50 mg/kg in combination with traztuzumab at 20 mg/kg both showed significant antitumor efficacy in subcutaneous colorectal cancer PDX xenograft model CR3056 in female BALB/c nude mice in this study.

### Example 3: In Vivo Efficacy Study of Tucatinib in the Treatment of HuPrime® PDX GA2140 Gastric Cancer Xenograft Model in Female BALB/c Nude Mice

The objective of this study was to evaluate preclinically the in vivo therapeutic efficacy of tucatinib in the treatment of the Huprime® PDX GA2140 gastric cancer xenograft model in female BALB/c nude mice. The Huprime® PDX GA2140 gastric cancer xenograft model has a L755S HER2 mutation.

Experiments and analysis were conducted as described in Example 1, with the exception of the Huprime® PDX GA2140 gastric cancer xenograft model being used instead of the Huprime® PDX GL1208 gallbladder cancer xenograft model.

### Results

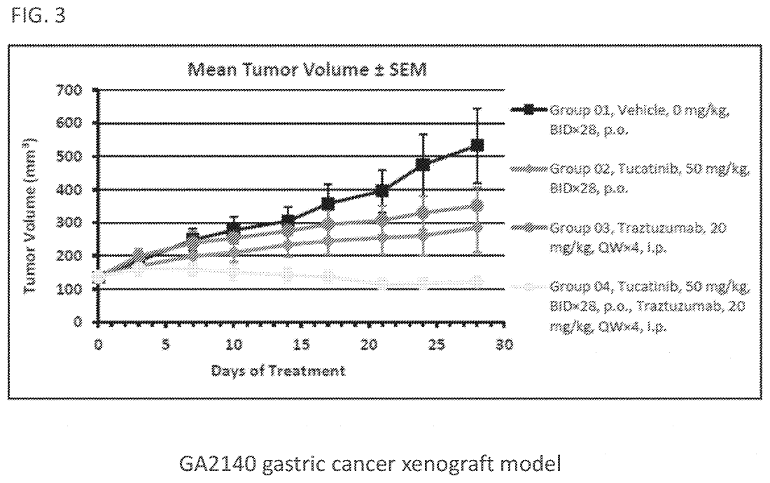

The mean tumor volume over time (±SEM) for each treatment group is shown in FIG. 3. Tumor growth inhibition (TGI) with data collected on day 28:

| | | Tumor size | TGI | T/C |
|---|---|---|---|---|
| Group | Treatment description | (mm³)^{a} on day 28 | (%)^{b} | (%)^{b} |
| 1 | Vehicle | 532.52 ± 113.30 | — | — |
| 2 | Tucatinib (50 mg/kg) | 284.53 ± 73.21 | 46.57 | 53.43 |
| 3 | Traztuzumab (20 mg/kg) | 350.77 ± 55.84 | 34.13 | 65.87 |
| 4 | Tucatinib (50 mg/kg) + | 121.16 ± 15.74 | 77.25 | 22.75 |
| | Traztuzumab (20 mg/kg) | | | |

Note:
^{a}Mean ± SEM;
^{b}Used mean Tumor Volume for TGI, and T/C calculation: T/C = mean (T28)/mean (C28) * 100%; TGI = (mean (C28) − mean (T28))/mean (C28) * 100%.

Statistical analysis of tumor volume with data collected on day 28:

| Test | P values | Significance level |
|---|---|---|
| Bartlett's test | 0.000387 | *** |
| Kruskal-Wallis test | 0.000822 | *** |
| G01-G02 | 0.0762 | ns |
| G01-G03 | 0.726 | ns |
| G01-G04 | 5.76e−05 | *** |
| G02-G03 | 0.456 | ns |
| G02-G04 | 0.0398 | * |
| G03-G04 | 0.000957 | *** |

Note:
ns = no significance

### Result Summary

In this study, the therapeutic efficacy of tucatinib as a single agent or in combination with trastuzumab in the treatment of subcutaneous gastric cancer PDX xenograft model GA2140 in female BALB/c nude mice was evaluated.

On day 28, neither the test compound tucatinib at 50 mg/kg (Group 2) nor traztuzumab at 20 mg/kg (Group 3) as single agents produced a statistical significant anti-tumor efficacy (TGI=46.57%, and 36.13%, respectively; both P>0.05) compared to vehicle treatment group (group 1).

However, on day 28, tucatinib at 50 mg/kg in combination with traztuzumab at 20 mg/kg (Group 4) produced a statistical significant anti-tumor efficacy (TGI=77.25%, and P <0.001) compared to vehicle treatment group (group 1).

In summary, tucatinib at 50 mg/kg in combination with traztuzumab at 20 mg/kg showed significant antitumor efficacy in subcutaneous gastric cancer PDX xenograft model GA2140 in female BALB/c nude mice in this study.

### Example 4: In Vivo Efficacy Study of Tucatinib in the Treatment of HuPrime® PDX GA6210 Gastric Cancer Xenograft Model in Female BALB/c Nude Mice

The objective of this study was to evaluate preclinically the in vivo therapeutic efficacy of tucatinib in the treatment of the Huprime® PDX GA6210 gastric cancer xenograft model in female BALB/c nude mice. The Huprime® PDX GA6210 gastric cancer xenograft model has an S310Y HER2 mutation.

Experiments and analysis were conducted as described in Example 1, with the exception of the Huprime® PDX gA6210 gastric cancer xenograft model being used instead of the Huprime® PDX GL1208 gallbladder cancer xenograft model.

### Results

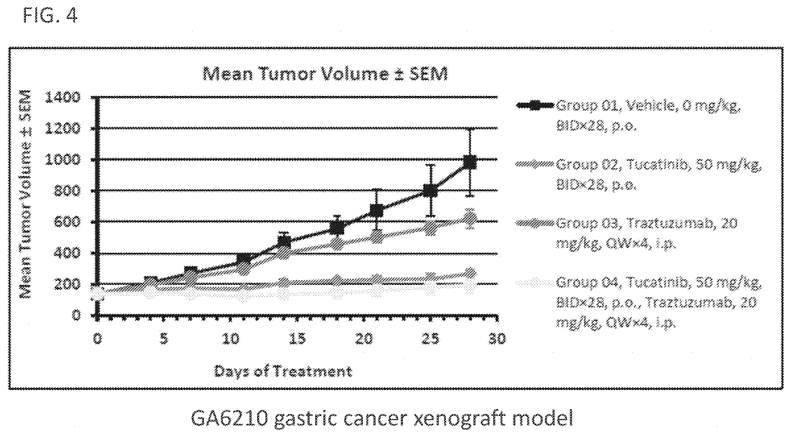

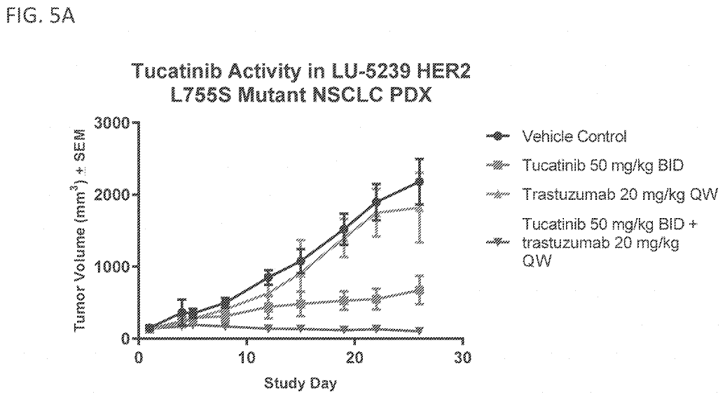

The mean tumor volume over time (±SEM) for each treatment group is shown in FIG. 4. Tumor growth inhibition (TGI) with data collected on day 28:

| | | Tumor size | TGI | T/C |
|---|---|---|---|---|
| Group | Treatment description | (mm³)^{a} on day 28 | (%)^{b} | (%)^{b} |
| 1 | Vehicle | 982.46 ± 214.83 | — | — |
| 2 | Tucatinib (50 mg/kg) | 265.69 ± 32.42 | 72.96 | 27.04 |
| 3 | Traztuzumab (20 mg/kg) | 620.64 ± 59.59 | 36.83 | 63.17 |
| 4 | Tucatinib (50 mg/kg) + | 192.80 ± 44.48 | 80.38 | 19.62 |
| | Traztuzumab (20 mg/kg) | | | |

Note:
^{a}Mean ± SEM;
^{b}Used mean Tumor Volume for TGI, and T/C calculation: T/C = mean (T28)/mean (C28) * 100%; TGI = (mean (C28) − mean (T28))/mean (C28) * 100%.

Statistical analysis of tumor volume with data collected on day 28:

| Test | P values | Significance level |
|---|---|---|
| Bartlett's test | 1.65e−06 | *** |
| Kruskal-Wallis test | 2.38e−05 | *** |
| G01-G02 | 8e−07 | *** |
| G01-G03 | 0.513 | ns |
| G01-G04 | 4.1e−08 | *** |
| G02-G03 | 3.22e−05 | *** |
| G02-G04 | 0.644 | ns |
| G03-G04 | 1.4e−06 | *** |

Note:
ns = no significance

### Result Summary

In this study, the therapeutic efficacy of tucatinib as a single agent or in combination with trastuzumabin the treatment of subcutaneous gastric cancer PDX xenograft model GA6210 in female BALB/c nude mice was evaluated.

On day 28, the test compound tucatinib at 50 mg/kg (Group 2) as a single agent or tucatinib at 50 mg/kg in combination with traztuzumab at 20 mg/kg (Group 4) both produced a statistical significant anti-tumor efficacy (TGI=72.96%, and 80.38%, respectively; both P <0.001) compared to vehicle treatment group (group 1).

However, on day 28, traztuzumab 20 mg/kg (Group 3) as a single agent didn't produce a statistical significant anti-tumor efficacy (TGI=36.83%, and P >0.05) compared to vehicle treatment group (group 1).

In summary, tucatinib at 50 mg/kg as a single agent or tucatinib at 50 mg/kg in combination with Traztuzumab at 20 mg/kg both showed significant antitumor efficacy in subcutaneous Gastric cancer PDX xenograft model GA6210 in female BALB/c nude mice in this study.

### Example 5: In Vivo Efficacy Study of Tucatinib in the Treatment of HuPrime® PDX LU-5239 Non-Small Cell Lung Cancer (NSCLC) Xenograft Model in Female BALB/c Nude Mice

The objective of this study was to evaluate preclinically the in vivo therapeutic efficacy of tucatinib in the treatment of the Huprime® PDX LU-5239 NSCLC cancer xenograft model in female BALB/c nude mice. The Huprime® PDX LU-5239 NSCLC cancer xenograft model has a L755S mutation in HER2.

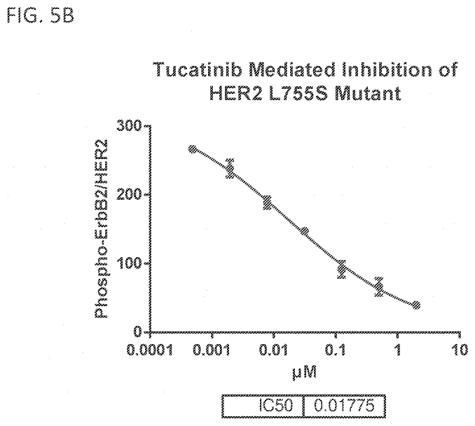

Experiments and analysis were conducted as described in Example 1, with the exception of the Huprime® PDX LU-5239 NSCLC cancer xenograft model being used instead of the Huprime® PDX GL1208 gallbladder cancer xenograft model. In addition, the ability of tucatinib to inhibit the kinase activity of HER2 L755S was assessed in vitro. As shown in FIG. 5B, tucatinib potently inhibits the kinase activity of the V777L activating mutation in vitro with an IC50 of 0.01775 μM.

### Results

The mean tumor volume over time (±SEM) for each treatment group is shown in FIG. 5A.

Statistical analysis of tumor volume with data collected on day 28:

| Test | P values | Significance level |
|---|---|---|
| G01-G02 | 0.0071 | ** |
| G01-G03 | 0.8255 | ns |
| G01-G04 | 0.0003 | *** |
| G02-G03 | 0.0464 | * |
| G02-G04 | 0.4591 | ns |
| G03-G04 | 0.0023 | ** |

Note:
ns = no significance

### Result Summary

In this study, the therapeutic efficacy of tucatinib as a single agent or in combination with trastuzumab in the treatment of subcutaneous NSCLC cancer PDX xenograft model LU-5239 in female BALB/c nude mice was evaluated.

On day 28, the test compound tucatinib at 50 mg/kg (Group 2) as a single agent or tucatinib at 50 mg/kg in combination with traztuzumab at 20 mg/kg (Group 4) both produced a statistical significant anti-tumor efficacy compared to vehicle treatment group (Group 1).

However, on day 28, traztuzumab 20 mg/kg (Group 3) as a single agent didn't produce a statistical significant anti-tumor efficacy compared to vehicle treatment group (Group 1).

In summary, tucatinib at 50 mg/kg as a single agent or tucatinib at 50 mg/kg in combination with traztuzumab at 20 mg/kg both showed significant antitumor efficacy in subcutaneous NSCLC cancer PDX xenograft model LU-5239 in female BALB/c nude mice in this study.

### Example 6: In Vivo Efficacy Study of Tucatinib in the Treatment of HuPrime® PDX CR-5085 Colorectal Xenograft Model in Female BALB/c Nude Mice

The objective of this study was to evaluate preclinically the in vivo therapeutic efficacy of tucatinib in the treatment of the Huprime® PDX CR-5085 colorectal cancer xenograft model in female BALB/c nude mice. The Huprime® PDX CR-5085 colorectal cancer xenograft model has a L755S mutation in HER2.

Experiments and analysis were conducted as described in Example 1, with the exception of the Huprime® PDX CR-5085 colorectal cancer xenograft model being used instead of the Huprime® PDX GL1208 gallbladder cancer xenograft model.

### Results

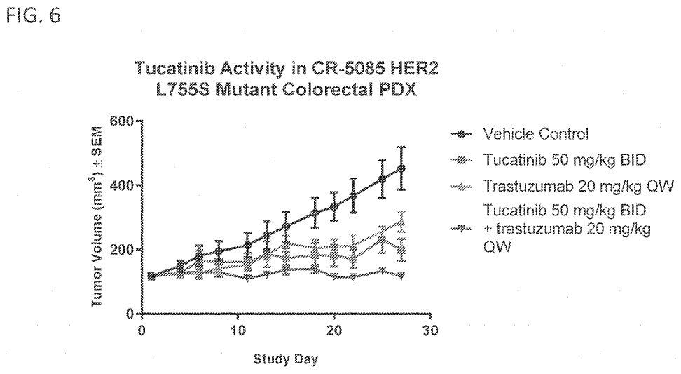

The mean tumor volume over time (±SEM) for each treatment group is shown in FIG. 6.

Statistical analysis of tumor volume with data collected on day 28:

| Test | P values | Significance level |
|---|---|---|
| G01-G02 | 0.0007 | *** |
| G01-G03 | 0.0331 | * |
| G01-G04 | <0.0001 | *** |
| G02-G03 | 0.4335 | ns |
| G02-G04 | 0.4765 | ns |
| G03-G04 | 0.0280 | * |

Note:
ns = no significance

### Result Summary

In this study, the therapeutic efficacy of tucatinib as a single agent or in combination with trastuzumab in the treatment of subcutaneous colorectal cancer PDX xenograft model CR-5085 in female BALB/c nude mice was evaluated.

On day 28, the test compounds tucatinib at 50 mg/kg (Group 2) and trastuzumab at 20 mg/kg (Group 3) as single agents or tucatinib at 50 mg/kg in combination with traztuzumab at 20 mg/kg (Group 4) produced a statistical significant anti-tumor efficacy compared to vehicle treatment group (Group 1).

In summary, tucatinib at 50 mg/kg as a single agent, trastuzumab at 20 mg/kg as a single agent, and tucatinib at 50 mg/kg in combination with traztuzumab at 20 mg/kg showed significant antitumor efficacy in subcutaneous colorectal cancer PDX xenograft model CR-5085 in female BALB/c nude mice in this study.

### Example 7: In Vivo Efficacy Study of Tucatinib in the Treatment of a Non-Small Cell Lung NSCLC Xenograft Model in Female BALB/c Nude Mice

The objective of this study was to evaluate preclinically the in vivo therapeutic efficacy of tucatinib in the treatment of a NSCLC cancer xenograft model having a HER2 G776insYVMA mutation in female BALB/c nude mice.

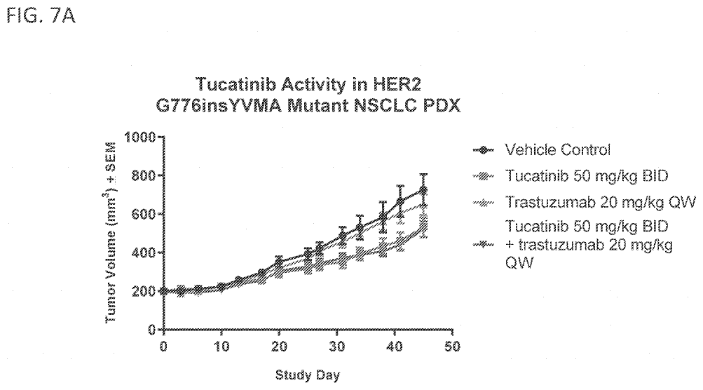

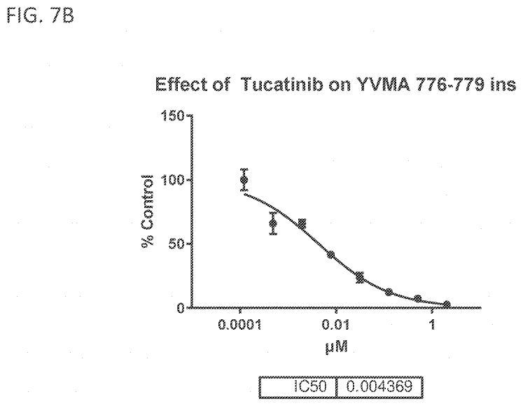

Experiments and analysis were conducted as described in Example 1, with the exception of the NSCLC cancer xenograft model having a HER2 G776insYVMA mutation being used instead of the Huprime® PDX GL1208 gallbladder cancer xenograft model. In addition, the ability of tucatinib to inhibit the kinase activity of HER2 G776insYVMA was assessed in vitro. As shown in FIG. 7B, tucatinib potently inhibits the kinase activity of the G776insYVMA HER2 insertion mutation in vitro with an IC50 of 0.004369 μM.

### Results

The mean tumor volume over time (±SEM) for each treatment group is shown in FIG. 7A.

### Result Summary

In this study, the therapeutic efficacy of tucatinib as a single agent or in combination with trastuzumab in the treatment of a NSCLC cancer xenograft model having a HER2 G776insYVMA mutation in female BALB/c nude mice was evaluated.

Although tucatinib demonstrated exceptional potency against the kinase activity of the YVMA (SEQ ID NO:2) insertion HER2 mutant, it showed limited activity in reducing tumor volume in this study.

What is claimed is:

1. A method for treating cancer in a subject comprising administering a therapeutically effective amount of tucatinib, or salt or solvate thereof, to the subject, wherein the cancer has been determined to express a mutant form of HER2.

2. A method for treating cancer in a subject comprising administering a therapeutically effective amount of tucatinib, or salt or solvate thereof, to the subject, wherein the cancer expresses a mutant form of HER2.

3. The method of claim 1 or claim 2, wherein the mutant form of HER2 is determined by DNA sequencing.

4. The method of claim 1 or claim 2, wherein the mutant form of HER2 is determined by determining RNA sequencing.

5. The method of any one of claims 1-4, wherein the mutant form of HER2 is determined by nucleic acid sequencing.

6. The method of claim 5, wherein the nucleic acid sequencing is next-generation sequencing (NGS).

7. The method of claim 1 or claim 2, wherein the mutant form of HER2 is determined by polymerase chain reaction (PCR).

8. The method of any one of claims 1-7, wherein the mutant form of HER2 is determined by analyzing a sample obtained from the subject.

9. The method of claim 8, wherein the sample obtained from the subject is a cell-free plasma sample.

10. The method of claim 8, wherein the sample obtained from the subject is a tumor biopsy.

11. The method of any one of claims 1-10, wherein the cancer does not have HER2 amplification, and wherein the absence of HER2 amplification is determined by immunohistochemistry (IHC).

12. The method of any one of claims 1-10, wherein the cancer has a HER2 amplification score of 0 or 1+, and wherein the HER2 amplification score is determined by immunohistochemistry (IHC).

13. The method of any one of claims 1-11, wherein the cancer has less than a 2 fold increase in HER2 protein levels.

14. The method of any one of claims 1-13, wherein the mutant form of HER2 comprises at least one amino acid substitution, insertion, or deletion compared to the amino acid sequence of SEQ ID NO:1.

15. The method of any one of claims 1-14, wherein the mutation in HER2 is an activating mutation.

16. The method of any one of claims 1-15, wherein the mutant form of HER2 comprises the amino acid substitution L755S.

17. The method of any one of claims 1-16, wherein the mutant form of HER2 comprises the amino acid substitution V777L.

18. The method of any one of claims 1-17, wherein the mutant form of HER2 comprises the amino acid substitution S310Y.

19. The method of any one of claims 1-18, wherein the mutant form of HER2 comprises a G776 YVMA insertion (G776 ins YVMA).

20. The method of any one of claims 1-19, wherein the cancer is selected from the group consisting of gastric cancer, colorectal cancer, lung cancer, gall bladder cancer, and breast cancer.

21. The method of claim 20, wherein the lung cancer is non-small cell lung cancer.

22. The method of claim 20, wherein the breast cancer is a HER2 positive breast cancer.

23. The method of any one of claims 1-22, wherein the tucatinib, or salt or solvate thereof, is administered to the subject at a dose of about 150 mg to about 650 mg.

24. The method of claim 23, wherein the tucatinib, or salt or solvate thereof, is administered to the subject at a dose of about 300 mg.

25. The method of claim 23 or 24, wherein the tucatinib, or salt or solvate thereof, is administered once or twice per day.

26. The method of claim 25, wherein the tucatinib, or salt or solvate thereof, is administered to the subject at a dose of about 300 mg twice per day.

27. The method of any one of claims 1-26, wherein the tucatinib is administered to the subject orally.

28. The method of any one of claims 1-27, further comprising administering one or more additional therapeutic agents to the subject to treat the cancer.

29. The method of claim 28, wherein the one or more additional therapeutic agents is selected from the group consisting of capecitabine and an anti-HER2 antibody.

30. The method of claim 28, wherein the one or more additional therapeutic agents is capecitabine.

31. The method of claim 28, wherein the one or more additional therapeutic agents is trastuzumab.

32. The method of claim 28, wherein the one or more additional therapeutic agents are capecitabine and trastuzumab.

33. The method of claim 30 or 32, wherein the capecitabine is administered to the subject at a dose of about 500 mg/m² to about 1500 mg/m².

34. The method of claim 33, wherein the capecitabine is administered to the subject at a dose of about 1000 mg/m².

35. The method of claim 33 or 34, wherein the capecitabine is administered to the subject orally.

36. The method of any one of claims 32-35, wherein the capecitabine is administered to the subject twice per day.

37. The method of claim 31 or 32, wherein the trastuzumab is administered to the subject at a dose of about 400 mg to about 800 mg.

38. The method of claim 37, wherein the trastuzumab is administered to the subject at a dose of about 600 mg.

39. The method of claim 37 or 38, wherein the trastuzumab is administered to the subject subcutaneously.

40. The method of claim 31 or 32, wherein the trastuzumab is administered to the subject intraperitoneally.

41. The method of claim 31 or 32, wherein the trastuzumab is administered to the subject at a dose of about 4 mg/kg to about 10 mg/kg.

42. The method of claim 41, wherein the trastuzumab is administered to the subject at a dose of about 6 mg/kg.

43. The method of claim 41, wherein the trastuzumab is administered to the subject at a dose of about 8 mg/kg.

44. The method of claim 41, wherein the trastuzumab is administered to the subject at an initial dose of about 8 mg/kg followed by subsequent doses of about 6 mg/kg.

45. The method of any one of claims 41-44, wherein the trastuzumab is administered intravenously.

46. The method of any one of claims 37-45, wherein the trastuzumab is administered once about every 1 week, once about every 2 weeks, once about every 3 weeks, or once about every 4 weeks.

47. The method of claim 46, wherein the trastuzumab is administered once about every 3 weeks.

48. The method of claim 47, wherein the tucatinib, capecitabine and trastuzumab are administered to the subject on a 21 day treatment cycle.

49. The method of claim 48, wherein the tucatinib is administered to the subject twice per day on each day of the 21 day treatment cycle.

50. The method of claim 48 or 49, wherein the capecitabine is administered to the subject twice per day on each of days 1-14 of the 21 day treatment cycle.

51. The method of any one of claims 48-50, wherein the trastuzumab is administered to the subject once per 21 day treatment cycle.

52. The method of claim 51, wherein the dose of trastuzumab during the first 21 day treatment cycle is 8 mg/kg and the dose of trastuzumab during the subsequent 21 day treatment cycles is 6 mg/kg.

53. The method of any one of claims 1-52, wherein treating the subject results in a tumor growth inhibition (TGI) index of at least about 85%.

54. The method of any one of claims 1-52, wherein treating the subject results in a TGI index of about 100%.

55. The method of any one of claims 1-54, wherein one or more therapeutic effects in the subject is improved after administration of tucatinib to the subject relative to a baseline.

56. The method of claim 55, wherein the one or more therapeutic effects is selected from the group consisting of: size of a tumor derived from the cancer, objective response rate, duration of response, time to response, progression free survival and overall survival.

57. The method of any one of claims 1-56, wherein the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of tucatinib to the subject.

58. The method of any one of claims 1-57, wherein the objective response rate is at least about 20%, at least about 25%, at least about 30%, at least about 35%, at least about 40%, at least about 45%, at least about 50%, at least about 60%, at least about 70%, or at least about 80%.

59. The method of any one of claims 1-58, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of tucatinib to the subject.

60. The method of any one of claims 1-59, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of tucatinib to the subject.

61. The method of any one of claims 1-60, wherein the duration of response to tucatinib is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of tucatinib to the subject.

62. The method of any one of claims 1-61, wherein the subject is a human.

63. Use of a therapeutically effective amount of tucatinib, or salt or solvate thereof, for the manufacture of a medicament for use in the method for treating cancer of any one of claims 1-62.

64. Tucatinib, or a salt or solvate thereof, for use in the method for treating cancer of any one of claims 1-62.

65. A pharmaceutical composition for treating cancer in a subject, the composition comprising tucatinib, or salt or solvate thereof, wherein the composition is for use in the method of any one of claims 1-62.

66. A kit comprising tucatinib, or salt or solvate thereof, and instructions for using the kit in the method of any one of claims 1-62.


SEQUENCE LISTING (2 sequences)

SEQ ID NO: 1 - protein, 1255 aa, Homo sapiens [sequence omitted]

SEQ ID NO: 2 - protein, 4 aa, Artificial Sequence
  Synthetic Construct
YVMA